(19) United States
(12) Patent Application Publication
(10) Pub. No.: US 2026/0109951 A1
(43) Pub. Date: Apr. 23, 2026

(54) METHODS AND COMPOSITIONS FOR INDUCING HEMATOPOIETIC CELL DIFFERENTIATION

(71) Applicant: FATE THERAPEUTICS, INC., San Diego, CA (US)

(72) Inventors: Alec WITTY, San Diego, CA (US); Amanda YZAGUIRRE, San Diego, CA (US); Jode GOODRIDGE, San Diego, CA (US); Bahram VALAMEHR, San Diego, CA (US); Samuel Adam LABARGE, San Diego, CA (US); Sean Sherman, San Diego, CA (US)

(21) Appl. No.: 19/111,618

(22) PCT Filed: Sep. 12, 2023
(86) PCT No.: PCT/US2023/074001

§ 371 Date: Mar. 13, 2025

Related U.S. Application Data
(60) Provisional application No. 63/375,680, filed on Sep. 14, 2022.

(51) Int. Cl. C12N 5/071; C07K 14/54; C12M 1/00; C12N 5/00

(52) U.S. Cl. CPC C12N 5/069; C07K 14/5443; C12M 47/04; C12N 5/525; C12N 2501/155; C12N 2501/727; C12N 2501/998; C12N 2506/45; C12N 2510/00

(57) ABSTRACT

In various aspects, the invention provides culture platforms, cell media, and methods of differentiating pluripotent cells into hematopoietic cells. In certain aspects, the invention further provides pluripotent stem cell-derived hematopoietic cells generated using the culture media and methods disclosed herein. The pluripotent stem cell-derived definitive HE cells produced by methods provided herein are capable of differentiating into hematopoietic lineage cells comprising T cell progenitors and T cells, in addition to NK cell progenitors, NK cells, NKT cells, or B cells.

### RELATED APPLICATION

This application claims priority to U.S. Provisional Application Ser. No. 63/375,680, filed on Sep. 14, 2022, the disclosure of which is hereby incorporated by reference in its entirety.

### FIELD OF INVENTION

The present disclosure relates generally to compositions and methods for manufacturing cells of all hematopoietic lineages from pluripotent stem cells. In particular aspects, the invention relates to improved culture platforms for manufacturing cells of all hematopoietic lineages from pluripotent stem cells including human induced pluripotent stem cells.

### BACKGROUND OF THE INVENTION

Human induced pluripotent stem cell (hiPSC) technology represents a highly promising and potentially unlimited source of therapeutically viable hematopoietic cells for the treatment of numerous hematological and non-hematological malignancies including cancer. To advance the promise of hiPSC and genomically engineered hiPSC technology as an allogeneic source of hematopoietic cellular therapeutics, it is essential to be able to efficiently and reproducibly generate not only hematopoietic stem and progenitor cells (HSCs) but also immune effector populations, including the diverse subsets of T, B, NKT, and NK lymphoid cells, and progenitor cells thereof.

The in vitro derivation of HSCs with the potential to generate lymphocytes is complicated by the existence of at least two temporally and spatially distinct waves of blood formation during embryonic development: primitive and definitive hematopoiesis. Primitive hematopoiesis initiates in the extraembryonic yolk sac and generates a transient and restricted hematopoietic repertoire mainly including primitive erythroid and myeloid cells. Nascent HSCs with the potential to generate lymphocytes only emerge later during the definitive wave from a specialized endothelial progenitor within the arterial vasculature termed definitive hemogenic endothelium (HE). Definitive HE then undergoes an endothelial-to-hematopoietic transition to give rise to HSCs, which then ultimately migrate to the bone marrow where they sustain multi-lineage hematopoiesis, including T, B, NKT, and NK lymphoid cells, throughout adult life. Therefore, the generation of HSCs and subsequently lymphoid effector cells from pluripotent stem cells is dependent upon the ability to accurately recapitulate the intricate stages of early embryonic hematopoietic development towards the definitive program through well-designed and validated methods and compositions.

A limited number of studies have described the directed differentiation of iPSCs to definitive HE in vitro. A major hurdle in utilizing hiPSCs for therapeutic purposes has been the requirement to initially co-culture such cells with murine- or human-derived stromal cells in the presence of ill-defined serum-containing media in order to maintain pluripotency and induce differentiation. In addition, the existing protocols have also employed a strategy consisting of culturing iPSC to form an embryoid body (EB), which is a heterogeneous aggregate of cells comprising various differentiated cells including ectoderm, mesoderm, and endoderm cells. Those procedures either require aggregating pluripotent cells by for example spinning to form clumps, allowing the cells to settle and aggregate in wells or allowing for passive aggregation and clump formation in suspension culture. The formed EBs are maintained for certain duration in differentiation inducing culture systems, typically seven to ten days, to allow for proper differentiation, then the EBs are either transferred to adherent culture for further maturation or dissociated into single cells for cell type selection in order to proceeding to the subsequent differentiation steps. (Kennedy et al., Cell Reports 2012:1722-1735; Knorr, et al., Stem Cells Translational Medicine 2013 (2): 274-283). For example, Kennedy et al. teach to generate EBs for iPSCs differentiation, where pluripotent cells were treated with collagenase and trypsin to allow for scraping of the cells to form small aggregates which were then cultured to form EBs. EB formation has been shown to facilitate pluripotent stem cell differentiation, however the requirement of forming aggregates and subsequent EBs is labor intensive, the cell numbers minimally increase in this process, the cellular content in the three dimensional EB aggregates are exposed to the media factors inconsistently and unevenly, which leads to heterogeneous cells products that are in variable differentiation stages, and greatly hinders the scalability and reproducibility of a manufacturing process that is required to be efficient and streamlined.

### SUMMARY OF THE INVENTION

In view of the foregoing, there is a need for methods and compositions to differentiate stem cells to definitive hematopoiesis efficiently and reliably without feeder co-culturing or relying on serum-containing media, and without requiring the formation of embryoid body aggregates as intermediates. Compositions and methods disclosed herein address this need, and provide other advantages as well.

In various aspects, the present invention relates generally to cell culture conditions, media, culture platforms, methods for culturing and differentiating stem cells to a hematopoietic cell fate, and to cell populations generated therefrom.

In some aspects, the present invention provides methods and compositions, including new cell surface markers identifying definitive hemogenic endothelium (HE) cells, for the generation of hematopoietic cell lineages derived from pluripotent stem cells, including iPSCs under serum/feeder-free conditions and in a scalable and monolayer culturing platform without the need of EB formation. Cells that may be differentiated according to methods disclosed herein range from pluripotent stem cells, to progenitor cells that are committed to a particular terminally differentiated cell and transdifferentiated cells, cells of various lineages directly transitioned to hematopoietic fate without going through a pluripotent intermediate. Similarly, the cells produced by differentiation of stem cells range from multipotent stem or progenitor cells to terminally differentiated stem cells, and all intervening hematopoietic cell lineages.

In one aspect, the invention provides a cell population comprising cells having a phenotype of: (i) CD82⁺; (ii) CD34⁺CD82⁺; and/or (iii) CD34⁺CD43⁻CD82⁺, wherein the cells comprise definitive hemogenic endothelium (HE) cells, and wherein the cells are derived from iPSC differentiation in vitro. In some embodiments of the cell population, the definitive HE cells are (i) enriched; and/or (ii) are capable of differentiating into hematopoietic lineage cells comprising T cell progenitors and T cells, in addition to NK cell progenitors, NK cells, NKT cells, or B cells. In some embodiments of the cell population, the iPSC is a clonal iPSC, a single cell dissociated iPSC, an iPSC cell line cell, or an iPSC master cell bank (MCB) cell. In some embodiments, wherein the iPSC is a naïve iPSC. In various embodiments of the cell population, the iPSC further comprises one or more genetic imprints introduced to the iPSC by genomic editing during or after reprogramming a non-pluripotent cell to the iPSC, wherein the genetic imprint comprises (i) one or more genetically modified modalities introduced through genomic insertion, deletion or substitution in the genome of the iPSC; or (ii) one or more retainable therapeutic attributes of a source specific immune cell that is donor-, disease-, or treatment response-specific, and wherein the iPSC is reprogrammed from the source specific immune cell; and wherein the cells comprise the same one or more genetic imprints.

In various embodiments of the cell population, the iPSC differentiation to obtain the cell population comprises: (i) differentiating iPSCs to obtain hemogenic endothelium (HE) cells, and (ii) sorting the HE cells for cells that are CD82⁺ (e.g., by using anti-CD82 antibodies) to obtain definitive HE cells expressing cell markers comprising CD82⁺, wherein the definitive HE cells are capable of differentiating into hematopoietic lineage cells. In some embodiments of the cell population, differentiating iPSCs to obtain HE cells further comprises: (a) differentiating iPSCs to obtain mesoderm progenitors; and (b) differentiating the mesoderm progenitors to obtain HE cells.

In some embodiments of the cell population, the cell markers further comprise CD34⁺, CD43⁺, RUNX1⁺, or any combinations thereof (e.g., obtained by sorting using an anti-CD34 antibody and/or an anti-CD43 antibody), wherein the obtained definitive HE cells comprise a phenotype of CD34⁺CD82⁺, CD34⁺CD43⁻CD82⁺, CD34⁺CD82⁺ RUNX1⁺, or CD34⁺CD43⁻CD82⁺RUNX1⁺. In various embodiments of the cell population, the iPSC differentiation comprises contacting the iPSCs with: (i) a cytokine that leads to a higher percentage of RUNX1 expressing cells as compared to without the cytokine; and/or (ii) a small molecule p38 MAPK (mitogen-activated protein kinase) inhibitor that leads to improved maintenance of CD82 expression in HE cells as compared to without the inhibitor. In some embodiments, the cytokine comprises BMP4 and/or the small molecule p38 MAPK inhibitor comprises DBM1285. In some embodiments, at least 0.5%, at least 1%, or at least 2% of the cells that are CD82⁺ are definitive HE cells. In some embodiments, the cell population is a substantially pure population of the cells having the phenotype (e.g., CD82⁺, CD34⁺CD82⁺, CD34⁺CD43⁻CD82⁺, CD34⁺CD82⁺RUNX1⁺, or CD34⁺CD43⁻CD82⁺RUNX1⁺).

In another aspect, the invention provides a composition comprising the cell population described herein. In some embodiments, the composition further comprises a cryopreservation medium.

In another aspect, the invention provides a method of generating iPSC-derived definitive HE, wherein the method comprises differentiating iPSC to obtain iPSC-derived hemogenic endothelium (HE) cells and sorting the HE cells for cells that are CD82⁺ (e.g., using antibodies comprising anti-CD82 antibody), thereby obtaining definitive HE cells expressing cell markers comprising CD82⁺, wherein the definitive HE cells are capable of differentiating into hematopoietic lineage cells comprising T cell progenitors and T cells, in addition to NK progenitors, NK cells, NKT cells, or B cells. In various embodiments of the method, the sorting further comprises sorting for cells that are CD34⁻, CD43⁻, RUNX1⁺, or any combinations thereof (e.g., obtained by sorting using an anti-CD34 antibody and/or an anti-CD43 antibody), wherein the obtained definitive HE cells comprise a phenotype of CD34⁺CD82⁺ or CD34⁺CD43⁻CD82⁺, CD34⁺CD82⁺RUNX1⁺, or CD34⁺CD43⁻CD82⁺RUNX1⁺. In various embodiments of the method, the method further comprises: (i) contacting the iPSCs with a medium comprising a BMP activator and bFGF, thereby differentiating the iPSCs to obtain mesoderm progenitors; and (ii) contacting the mesoderm progenitors with a medium comprising a BMP activator, bFGF, VEGF, a Wnt pathway activator and optionally a p38 MAPK inhibitor, thereby differentiating the mesoderm progenitors to obtain HE cells. In various embodiments of the method, contacting with the p38 MAPK inhibitor increases maintenance of CD82 expression in HE cells as compared to without the p38 MAPK inhibitor; the BMP activator comprises BMP4; and/or the Wnt pathway activator comprises a GSK3 inhibitor. In some embodiments, the p38 MAPK inhibitor comprises DBM1285; and/or the GSK3 inhibitor comprises CHIR99021.

In some embodiments of the method of generating iPSC-derived definitive HE, the iPSCs comprise naïve iPSCs, and/or are derived from iPSCs comprising one or more genetic imprints. In some embodiments, the one or more genetic imprints comprised in the iPSCs are retained in the iPSC-derived definitive HE cells. In various embodiments, the method further comprises cryopreserving the definitive HE cells.

In another aspect, the invention provides a composition for generating iPSC-derived definitive HE (hemogenic endothelium) cells comprising: a BMP activator, bFGF, VEGF, a Wnt pathway activator, and optionally a p38 MAPK inhibitor. In various embodiments of the composition, (i) the composition is free of TGFβ receptor/ALK inhibitors; (ii) the generated iPSC-derived definitive HE comprises increased RUNX1-expressing cells as compared to differentiation without the BMP activator; and/or (iii) the generated iPSC-derived definitive HE comprises increased CD82-expressing cells as compared to differentiation without the p38 MAPK inhibitor. In some embodiments of the composition, the BMP activator comprises BMP4; and/or the p38 MAPK inhibitor comprises at least one of DBM1285, VX-745, VX-702, RO-4402257, SCIO-469, BIRB-796, SD-0006, PH-797804, AMG-548, LY2228820, SB-681323, GW-856553, RV568, CAS 219138-24-6, SB203580, and SB242235. In some embodiments of the composition, the p38 MAPK inhibitor comprises DBM1285. In some embodiments, the composition further comprises iPSCs, mesodermal cells, or the definitive HE cells.

In another aspect, the invention provides a method of generating iPSC-derived definitive HE comprising: (i) differentiating iPSCs to obtain mesoderm progenitor cells; (ii) differentiating the mesoderm progenitor cells to obtain HE cells; and (iii) sorting the HE cells for cells that are CD82⁺ (e.g., by using anti-CD82 antibodies) to obtain definitive hemogenic endothelium (HE) cells expressing cell markers comprising CD82⁺, wherein the definitive HE cells are capable of differentiating into hematopoietic lineage cells comprising T cell progenitors and T cells, in addition to NK cell progenitors, NK cells, NKT cells, or B cells. In various embodiments of the method, the sorting further comprises sorting for cells that are CD34⁺, CD43⁺, RUNX1⁺, or any combinations thereof (e.g., obtained by sorting using an anti-CD34 antibody and/or an anti-CD43 antibody), and wherein the obtained definitive HE cells comprise a phenotype of CD34⁺CD82⁺, CD34⁺CD43⁻CD82⁺, CD34⁺CD82⁺RUNX1⁺, or CD34⁺CD43⁻CD82⁺RUNX1⁺. In some embodiments of the method, step (ii) of differentiating the mesoderm progenitor cells to HE comprises contacting the mesoderm progenitor with: (i) a cytokine that leads to a higher percentage of RUNX1 expressing HE cells as compared to without the cytokine; and/or (ii) a small molecule p38 MAPK (mitogen-activated protein kinase) inhibitor that leads to improved maintenance of CD82 expression in HE cells as compared to without the inhibitor. In some embodiments, the cytokine comprises BMP4 and/or the small molecule p38 MAPK inhibitor comprises DBM1285. In some embodiments, the method further comprises cryopreserving the obtained definitive HE cells.

In another aspect, the invention provides a method of generating iPSC-derived hematopoietic lineage cells by differentiating the definitive HE cells described herein, wherein the method comprises contacting the definitive HE cells with a medium composition comprising SCF, Flt3L, and IL7; and optionally one or more of a ROCK inhibitor, TPO, and IL3, thereby obtaining the iPSC-derived hematopoietic lineage cells comprising T cell progenitors and T cells, in addition to NK cell progenitors, NK cells, NKT cells, or B cells. In various embodiments of the method, the iPSC-derived hematopoietic lineage cells comprise NK cell progenitors, and/or NK cells, and wherein (1) the medium composition further comprises IL15; and/or (2) the definitive HE cells comprise a genetic insertion of a polynucleotide encoding a cytokine signaling complex comprising a partial or full peptide of cell surface expressed exogenous IL 15 and/or a receptor thereof. In some embodiments of the method, the medium composition is free of OP9 stromal cells. In some embodiments, the differentiating occurs in the presence of an extracellular matrix comprising a recombinant human fibronectin or fragment thereof and Fc-rhDLL4 (human DLL4 Fc chimera recombinant protein).

In another aspect, the invention provides a method of manufacturing iPSC-derived hematopoietic lineage cells comprising: differentiating iPSCs to obtain definitive hemogenic endothelium (HE) cells, wherein the definitive HE cells express cell markers comprising CD82⁺; and differentiating the definitive HE cells to obtain iPSC-derived hematopoietic lineage cells; wherein the iPSC-derived hematopoietic lineage cells comprise T cell progenitors and T cells, in addition to NK cell progenitors, NK cells, NKT cells, or B cells. In some embodiments of the method, the iPSCs comprise one or more genetic imprints introduced to the iPSCs by genomic editing during or after reprogramming non-pluripotent cells to the iPSCs, wherein the one or more genetic imprints comprise: (i) one or more genetically modified modalities introduced through genomic insertion, deletion or substitution in the genome of the iPSCs; or (ii) one or more retainable therapeutic attributes of source specific immune cells that are donor-, disease-, or treatment response-specific, wherein the iPSCs are reprogrammed from the source specific immune cells, and wherein the one or more genetic imprints is retained in the iPSC-derived hematopoietic lineage cells. In some embodiments of the method, differentiating the iPSCs to obtain definitive hemogenic endothelium (HE) cells comprises: (i) differentiating the genetically engineered iPSCs to obtain mesoderm progenitors; (ii) differentiating the mesoderm progenitors to obtain HE cells; and (iii) sorting the HE cells for cells that are CD82⁺ (e.g., by using anti-CD82 antibodies) to obtain the definitive HE cells expressing cell markers comprising CD82⁺. In some embodiments, the sorting further comprises sorting for cells that are CD34⁺, CD43⁻, RUNX1⁺, or any combinations thereof (e.g., obtained by sorting using an anti-CD34 antibody and/or an anti-CD43 antibody), and wherein the obtained definitive HE cells comprise a phenotype of CD34⁺CD82⁺, CD34⁺CD43⁻CD82⁺, CD34⁺CD82⁺RUNX1⁺, or CD34⁺CD43⁻CD82⁺RUNX1⁺.

In various embodiments of the method, differentiating the mesoderm progenitor to HE cells comprises contacting the mesoderm progenitor with: (i) a cytokine that leads to a higher percentage of RUNX1 expressing HE cells as compared to without the cytokine; and/or (ii) a small molecule p38 MAPK (mitogen-activated protein kinase) inhibitor that leads to improved maintenance of CD82 expression in HE cells as compared to without the inhibitor. In some embodiments, the cytokine comprises a BMP activator (e.g., BMP4) and/or the small molecule p38 MAPK inhibitor comprises DBM1285. In some embodiments, the method further comprises cryopreserving the definitive HE cells, wherein the cryopreserved definitive HE cells are thawed prior to differentiation thereof. In some embodiments, differentiating the definitive HE cells is free of OP9 stromal cells. In some embodiments, differentiating the definitive HE cells occurs in the presence of an extracellular matrix comprising a recombinant human fibronectin or fragment thereof and Fc-rhDLL4.

In another aspect, the invention provides a method of generating an NK cell in a feeder-free environment comprising: (a) differentiating an iPSC or definitive HE cell derived therefrom to NK lineage cells in a culture medium comprising one or more growth factors and cytokines comprising SCF, Flt3L, and IL7; wherein the culture medium is free of OP9 stromal cells; and further wherein: (i) the culture medium comprises IL15, and/or (ii) the definitive HE cells comprise a genetic insertion of a polynucleotide encoding a cytokine signaling complex comprising a partial or full peptide of cell surface expressed exogenous IL15 and/or a receptor thereof; and (b) expanding and activating the NK lineage cells to obtain NK cells having cytotoxicity against a target. In some embodiments, the culture medium further comprises one or more of a ROCK inhibitor, TPO, and IL3. In some embodiments, the definitive HE cell comprises a phenotype comprising: (i) CD82⁺; (ii) CD34⁺CD82⁺; (iii) CD34⁺CD43⁻CD82; and/or (iv) CD34⁺, and at least one of CD43⁻, CD93⁻, CXCR4⁻, CD73⁻, and RUNX1⁺.

In some embodiments of the method of generating an NK cell, differentiating the iPSC further comprises: (i) differentiating the iPSC to obtain mesoderm progenitors; (ii) differentiating the mesoderm progenitors to obtain hemogenic endothelium (HE) cells; and (iii) sorting HE cells for cells that are CD82⁺ (e.g., by using anti-CD82 antibodies) to obtain the definitive HE cells, wherein the definitive HE cell expresses cell markers comprising CD82⁺. In some embodiments of the method, the sorting further comprising sorting for cells that are CD34⁺, CD43⁻, RUNX1⁺, or any combinations thereof (e.g., obtained by sorting using an anti-CD34 antibody and/or an anti-CD43 antibody), wherein the obtained definitive HE cells comprise a phenotype of CD34⁺CD82⁺, CD34⁺CD43⁻CD82⁺, CD34⁺CD82⁺ RUNX1⁺, or CD34⁺CD43⁻CD82⁺RUNX1⁺. In some embodiments, differentiating the mesoderm progenitor to HE cells comprises contacting the mesoderm progenitor with: (i) a cytokine that leads to a higher percentage of RUNX1 expressing HE cells as compared to without the cytokine; and/or (ii) a small molecule p38 MAPK (mitogen-activated protein kinase) inhibitor that leads to improved maintenance of CD82 expression in HE cells as compared to without the inhibitor. In some embodiments, the cytokine comprises BMP4 and/or the small molecule p38 MAPK inhibitor comprises DBM1285.

In some embodiments of the method, step (a) differentiating further comprises contacting the definitive HE cells with an extracellular matrix comprising a recombinant human fibronectin or fragment thereof and Fc-rhDLL4; and/or step (b) expanding further comprises contacting the NK lineage cells with an expansion composition comprising nicotinamide. In some embodiments, step (b) expanding further comprises contacting the NK lineage cells with a small molecule AhR inhibitor, thereby modulating the NK lineage cell activation. In some embodiments, the small molecule AhR inhibitor comprises CHIR223191, UM729, UM171, or SR1.

### BRIEF DESCRIPTION OF THE DRAWINGS

FIG. 1 shows flow cytometric analysis of D10 cells differentiated under control conditions and with cytokines that promote the specification of RUNX1⁺ HE (cytokine driven).

FIG. 2 shows a summary of candidate HE surface markers identified via a BioLegend LEGENDScreen.

FIG. 3A shows UMAP visualization of the expression of curated genes used for the identification of cell clusters.

FIG. 3B shows transcriptomic identification of D10 cell populations visualized by UMAP. Each dot represents one cell.

FIG. 3C shows a Violin plot of CD82 expression within each cell cluster from FIG. 3B.

FIGS. 4A-4B show flow cytometric analysis of cytokine driven D10 cells comparing expression of HE candidate markers with RUNX1 and CD82.

FIG. 5 shows flow cytometric analysis of cytokine driven D10 cells demonstrating enrichment of CD82⁺ cells within the CD73⁻CD93⁻CXCR4⁻ endothelial population. Cells are pre-gated on single/live events.

FIG. 6 shows frequency of HE within cytokine driven D10 populations that were fluorescence-activated cell sorted (FACs) based on indicated markers on x-axis (mean±SD).

FIG. 7 shows flow cytometric analysis of D35 iT cells derived from cytokine driven D10 populations FAC sorted based on the markers indicated above flow plots.

FIG. 8 shows flow cytometric analysis of D30 iNK cells derived from cytokine driven D10 populations FAC sorted based on the markers indicated above flow plots.

FIGS. 9A-9D show a comparison of the fold expansion and iNK specification between cells differentiated on Retro/DLL4, irOP9-DLL4 and a commercially available kit.

FIGS. 10A-10C show a comparison of the fold expansion of progenitor iNK cells obtained using each of the three differentiation strategies when co-cultured with engineered feeder cells to obtain activated NK cells.

FIGS. 11A and 11B show that antigen-dependent caspase 3/7 activity is comparable between mature iNK cells differentiated on Retro/DLL4 or irOP9-DLL4.

FIGS. 12A and 12B show that IFNγ and TNFα cytokine release are comparable between mature iNK cells differentiated on Retro/DLL4 or irOP9-DLL4.

FIG. 13 shows that iNK cells differentiated on Retro/DLL4 display similar antigen-dependent serial killing as cells differentiated on irOP9-DLL4.

FIG. 14 shows that addition of an AhR (aryl hydrocarbon receptor) inhibitor during the cell expansion stage of iPSC differentiation results in a greater fold expansion and yield of differentiated NK cells.

FIG. 15 shows that cells treated with an AhR inhibitor prior to cryopreservation showed enhanced post thaw anti-tumor efficacy over time.

### DETAILED DESCRIPTION OF THE INVENTION

In various aspects, the invention generally relates to methods and compositions for differentiating stem cells toward a definitive hematopoietic cell fate. In particular aspects, the invention provides a multi-stage differentiation platform wherein iPSC or iPSC-derived cells at various stages of development can be induced to assume a definitive hematopoietic phenotype, ranging from definitive hemogenic endothelium, to fully differentiated hematopoietic cells including, T cells, B cells, NKT cells, and NK cells. Methods and compositions are provided for making a cell more susceptible to assuming a definitive hematopoietic fate, for example, a CD34⁺ definitive hematopoietic stem cell. In some embodiments, the method and compositions of the present invention generate definitive hemogenic endothelium (HE) from naïve iPSCs in a scalable manner by avoiding the formation of EBs or aggregates.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, or both, or any combination thereof of the alternatives.

The term “and/or” should be understood to mean either one, or both of the alternatives.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

The term “effector cell” generally is applied to certain cells in the immune system that carry out a specific activity in response to stimulation and/or activation, or to cells that effect a specific function upon activation. As used herein, the term “effector cell” includes, and in some contexts is interchangeable with, immune cells, “differentiated immune cells,” and primary or differentiated cells that are edited and/or modulated to carry out a specific activity in response to stimulation and/or activation. Non-limiting examples of effector cells include primary-sourced or iPSC-derived T cells, NK cells, NKT cells, B cells, macrophages, and neutrophils.

As used herein, the terms “B lymphocyte” or “B cell” are used interchangeably and refer to a subset of lymphocytes defined by the expression of a B cell receptor comprised of immunoglobulin heavy and light chains (BCR, Ig), CD19 or CD20, in absence of the T cell receptor (CD3). As provided herein, the B cell can also be derived from a stem or progenitor cell via directed differentiation. The B cell comprises any subtype of B cell, and can be of any developmental stage, including but not limited to, pro-B cells, pre-B cells, naïve B cells, B-1 B cell, B-2 B cell, marginal zone B cells, follicular B cells, memory B cells, plasmablast cells, plasma cells, regulatory B cells.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells in an MHC class I-restricted manner. A T cell can be any T cell, such as a cultured T cell, e.g., a primary T cell, or a T cell from a cultured T cell line, e.g., Jurkat, SupT1, etc., or a T cell obtained from a mammal. The T cell can be a CD3⁺ cell. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The term “T cell” can also refer to a genetically engineered T cell, such as a T cell modified to express a T cell receptor (TCR) or a chimeric antigen receptor (CAR). A T cell or T cell like effector cell can also be differentiated from a stem cell or progenitor cell (“a derived T cell” or “a derived T cell like effector cell”, or collectively, “a derivative T lineage cell”). A derived T cell like effector cell may have a T cell lineage in some respects, but at the same time has one or more functional features that are not present in a primary T cell. In this application, a T cell, a T cell like effector cell, a derived T cell, a derived T cell like effector cell, or a derivative T lineage cell, are collectively termed as “a T lineage cell”.

“CD4⁺ T cells” refers to a subset of T cells that express CD4 on their surface and are associated with cell-mediated immune response. They are characterized by secretion profiles following stimulation, which may include secretion of cytokines such as IFN-gamma, TNF-alpha, IL2, IL4 and IL10. “CD4” molecules are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refer to a subset of peripheral blood lymphocytes defined by the expression of CD56 or CD16 and the absence of the T cell receptor (CD3). An NK cell can be any NK cell, such as a cultured NK cell, e.g., a primary NK cell, or an NK cell from a cultured or expanded NK cell or a cell-line NK cell, e.g., NK-92, or an NK cell obtained from a mammal that is healthy or with a disease condition. As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression. An NK cell, or an NK cell like effector cell may be differentiated from a stem cell or progenitor cell (“a derived NK cell” or “a derived NK cell like effector cell”, or collectively, “a derivative NK lineage cell”). A derivative NK cell like effector cell may have an NK cell lineage in some respects, but at the same time has one or more functional features that are not present in a primary NK cell. In this application, an NK cell, an NK cell like effector cell, a derived NK cell, a derived NK cell like effector cell, or a derivative NK lineage cell, are collectively termed as “an NK lineage cell”.

As used herein, the term “NKT cells” or “natural killer T cells” or “NKT lineage cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified by the expression of one or more of the following markers: TCR Va24-Ja18, Vb11, CD1d, CD3, CD4, CD8, aGalCer, CD161 and CD56.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors. In some embodiments, a population of iHE cells may be maintained, stored, and/or cryopreserved in multiple vessels to reliably serve as the starting cellular material for the production of cell-based therapeutics through directed differentiation in manufacturing settings.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T lineage cells, NK lineage cells and B lineage cells. Hematopoietic cells also include various subsets of primitive hematopoietic cells that give rise to primitive erythrocytes, megakarocytes and macrophages.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta and are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

As used herein, the term “pluripotent” refers to the ability of a cell to form all lineages of the body or soma (i.e., the embryo proper). For example, embryonic stem cells are a type of pluripotent stem cells that are able to form cells from each of the three germ layers: the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs” means that the stem cells are produced from differentiated adult, neonatal or fetal cells that have been induced or changed (i.e., reprogrammed) into cells capable of differentiating into tissues of all three germ or dermal layers: mesoderm, endoderm, and ectoderm.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60, TRA1-81, TRA1-85, TRA2-54, GC™-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “naïve” or “ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed state of pluripotency. Under standard pluripotent cell culture conditions such cells remain in the primed state unless the exogenous transgene expression is maintained, wherein characteristics of the ground state are observed.

Pluripotency exists as a continuum and induced pluripotent stem cells (iPSCs) appear to exist in both the “primed” state and the “naïve” state, with a cell in the naïve state possibly having greater differentiation potential. Induced pluripotent stem cells generated in conventional culture medium exist in a primed state and more closely resemble cells derived from a post-implantation blastocyst, while naïve iPSCs display pluripotency characteristics that more closely resemble mouse embryonic stem cells or cells derived from a pre-implantation blastocyst. The primed and naïve cell states can be defined by various differences, including differences in colony morphology, cellular response to inhibition or activation of key signaling pathways, gene expression signature, and ability to reactivate genes associated with extraembryonic cells. For example, conventional iPSCs, representing a primed pluripotent state, exhibit a colony morphology that is flat, while naïve iPSCs exhibit a compact domed colony morphology that is similar to mouse embryonic stem cells. As used herein, the term “pluripotent stem cell morphology” refers to the classical morphological features of an embryonic stem cell. Normal embryonic stem cell morphology is characterized by being round and compact in shape, with a high nucleus-to-cytoplasm ratio, the notable presence of nucleoli, and typical inter-cell spacing.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (i.e., ectoderm, mesoderm and endoderm germ layers) with modest proliferation within the EB cell cluster. Although proven to facilitate cell differentiation, EBs, however, give rise to heterogeneous cells in variable differentiation states because of the inconsistent exposure of the cells in the three-dimensional structure to the differentiation cues within the environment. In addition, EBs are laborious to create and maintain. Moreover, cell differentiation through EB formation is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates, forming larger aggregates, which can be mechanically or enzymatically dissociated into smaller aggregates to maintain cell proliferation within the culture and increase numbers of cells. As distinct from EB culture, cells cultured within aggregates in maintenance culture media maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “embryoid bodies”, “EBs”, or “EB formation.” Monolayer differentiation, among other advantages disclosed herein, avoids the need for EB formation to initiate differentiation. Because monolayer culturing does not mimic embryo development such as is the case with EB formation, differentiation towards specific lineages is deemed to be minimal as compared to all three germ layer differentiation in EB formation.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder or stromal cells and is also not pre-conditioned by the cultivation of feeder cells. Feeder cells include, but without limitation, stromal cells, mouse embryonic fibroblasts, human fibroblasts, keratinocytes, and embryonic stem cells.

“Cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, “passage” or “passaging” refers to the act of splitting the cultured cells by subdividing and plating cells into multiple cell culture surfaces or vessels when the cells have proliferated to a desired extent. In some embodiments “passage” or “passaging” refers to subdividing, diluting and plating the cells. As cells are passaged from the primary culture surface or vessel into a subsequent set of surfaces or vessels, the subsequent cultures may be referred to herein as “secondary culture” or “first passage,” etc. Each act of subdividing and plating into a new culture vessel is considered one passage. In some embodiments, the cultured cells are passaged every 1, 2, 3, 4, 5, 6, 7, or more, days. In some embodiments, the initially selected iPSCs after reprogramming are passaged once every 3-7 days.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be enzymatically or mechanically dissociated from each other, such as by dissociation into a suspension of clusters, single cells or a mixture of single cells and clusters. In yet another alternative embodiment, adherent cells can be dissociated from a culture plate or other surface. Dissociation thus can involve breaking cell interactions with extracellular matrix (ECM) and substrates (e.g., culture surfaces), or breaking the ECM between cells.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated from a cell culture or cell suspension. Therefore, an “isolated cell” is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained by separating the desired cells, or populations thereof, from other substances or cells in the environment, or by removing one or more other cell populations or subpopulations from the environment.

As used herein, the term “purify” or the like refers to increasing purity. For example, the purity of a specific cell type within a population of cells can be increased to at least 50%, 60%, 70%, 80%, 90%, 95%, 99%, or 100%.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

As used herein, the terms “reprogramming” or “dedifferentiation” or “increasing cell potency” or “increasing developmental potency” refer to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state. A “reprogramming cell,” as opposed to a “reprogrammed cell,” refers to a non-pluripotent cell undergoing reprogramming/dedifferentiation toward a pluripotent state, presenting a transitional morphology (i.e., a change in morphology) yet without the hallmarks of a pluripotent cell, including pluripotent stem cell morphology or stable endogenous pluripotency gene expression such as OCT4, NANOG, SOX2, SSEA4, TRA181, CD30 and/or CD50. The transitional morphology of a “reprogramming cell” distinguishes the cell from the starting non-pluripotent cell prior to reprogramming induction, as well as from a reprogrammed cell having the embryonic stem cell hallmark morphology. For example, when reprogramming a fibroblast, the morphological change of the reprogramming cell comprises MET (mesenchymal to epithelial transition). A person skilled in the art readily understands and identifies such transitional morphology for various types of somatic cells induced to reprogram. In some embodiments, the reprogramming cells are intermediary cells that have been induced to reprogram for at least 1, 2, 3, 4, 5, 6, 7, 8, or more days, but no more than 21, 22, 24, 26, 28, 30, 32, 35, 40 days or any number of days in between, wherein the cells have not entered a self-maintaining or self-sustaining pluripotent state. A non-pluripotent cell is induced to reprogram when the cell is introduced with one or more reprogramming factors. A reprogramming cell that has been induced to reprogram for 1, 2, 3, or 4 days is a cell 1, 2, 3, or 4 days post transduction of the reprogramming factors (the day of transduction is day 0). Unlike the somatic cell prior to the exposure to the exogenous expression of reprogramming factors, a “reprogramming cell” can progress within the reprogramming process to reach a stable pluripotent state and becomes a “reprogrammed cell” even without the presence of the exogenous expression reprogramming factors, so long as a sufficient time period is given.

A “pluripotency factor” or “reprogramming factor” refers to an agent or a combination of agents used for inducing or increasing the developmental potency of a cell. Pluripotency factors include, without limitation, polynucleotides, polypeptides, and small molecules capable of increasing the developmental potency of a cell. Exemplary pluripotency factors include, for example, transcription factors OCT4 and SOX2, and small molecule reprogramming agents such as, for example, TGFβ inhibitor, GSK3 inhibitor, MEK inhibitor and ROCK inhibitor.

As used herein, “genetic modification” refers to genetic editing including those (1) naturally derived from rearrangements, mutations, genetic imprinting and/or epigenetic modification that take place in a cell or in cell development, or (2) obtained through genomic engineering through cell manipulation including, but not limited to, insertion, deletion or substitution in the genome of a cell. Genetic modification, as used herein, also includes one or more retainable therapeutic attributes of a source-specific immune cell that is donor-, disease-, or treatment response-specific. Genetically modified cells are cells comprising the genetic modification (e.g., a genetic edit) as compared to corresponding wildtype cells that do not have such genetic modification.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level-successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level—successful removal, addition, or alteration of a cell function/characteristic via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at iPSC, progenitor or dedifferentiated cellular origin.

As used herein, the term “genetic imprint” refers to genetic or epigenetic information that contributes to preferential and/or enhanced therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK cells or derivative T lineage cells, as used throughout this application are cells differentiated from an iPSC, as compared to their primary counterparts obtained from natural/native sources such as peripheral blood, umbilical cord blood, or other donor tissues. As used herein, the genetic imprint(s) conferring a preferential and/or enhanced therapeutic attribute is incorporated into the iPSCs either through reprogramming a selected source cell that is donor-, disease-, or treatment response-specific, or through introducing genetically modified modalities to iPSCs using genomic editing. In the aspect of a source cell obtained from a specifically selected donor, disease or treatment context, the genetic imprint contributing to preferential therapeutic attributes may include any context-specific genetic or epigenetic modifications which manifest a retainable phenotype, i.e., a preferential therapeutic attribute, that is passed on to iPSC-derived cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential and/or enhanced therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; and resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. In general, a synthetic cell possesses one or more non-native cell functions when compared to its closest counterpart primary cell, whether the synthetic cell is differentiated from engineered pluripotent cells or obtained by engineering a primary cell from natural/native sources, such as peripheral blood, umbilical cord blood, or other donor tissues. For example, synthetic effector cells, or synthetic NK cells or synthetic T cells, as used throughout this application are cells differentiated from a genomically modified iPSC, as compared to their primary counterpart obtained from natural/native sources such as peripheral blood, umbilical cord blood, or other donor tissues. In some embodiments, the synthetic cell possesses one or more non-native cell functions when compared to its closest counterpart primary cell.

As used herein, the term “exogenous” is intended to mean that the referenced molecule or the referenced activity is introduced into, or is non-native to, the host cell. The exogenous molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. Thus, the term “vector” comprises the construct to be delivered. A vector can be a linear or a circular molecule. A vector can be integrating or non-integrating. The major types of vectors include, but are not limited to, plasmids, episomal vectors, viral vectors, cosmids, and artificial chromosomes. Viral vectors include, but are not limited to, adenovirus vectors, adeno-associated virus vectors, retrovirus vectors, lentivirus vectors, Sendai virus vectors, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as “encoding” the protein or other product of that gene or cDNA.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. “Polynucleotide” also refers to both double- and single-stranded molecules.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

“Fusion proteins” or “chimeric proteins”, as used herein, are proteins created through genetic engineering to join two or more partial or whole polynucleotide sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added.

As used herein, the term “engager” refers to a molecule, e.g., a fusion polypeptide, which is capable of forming a link between an immune cell (e.g., a T cell, a NK cell, a NKT cell, a B cell, a macrophage, a neutrophil), and a tumor cell; and activating the immune cell. Examples of engagers include, but are not limited to, bi-specific T cell engagers (BiTEs), bi-specific killer cell engagers (BiKEs), tri-specific killer cell engagers (TrikEs), or multi-specific killer cell engagers, or universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, a NK cell, a NKT cell, a B cell, a macrophage, or a neutrophil. In some embodiments, the surface triggering receptor facilitates bi- or multi-specific antibody engagement between the effector cells and a specific target cell (e.g., a tumor cell) independent of the effector cells' natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some cases, and to kill two or more types of tumors in other cases. A surface triggering receptor generally comprises a co-stimulatory domain for effector cell activation and an anti-epitope that is specific to the epitope of an engager. A bi-specific engager is specific to the anti-epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch protein could mediate induction of apoptosis, inhibition of protein synthesis, DNA replication, growth arrest, transcriptional and post-transcriptional genetic regulation and/or antibody-mediated depletion. In some instances, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing, curative or palliative properties against a disease and may be administered to ameliorate, relieve, alleviate, reverse or lessen the severity of a disease. A pharmaceutically active protein also has prophylactic properties and is used to prevent the onset of a disease or to lessen the severity of such disease or pathological condition when it does emerge. “Pharmaceutically active proteins” include an entire protein or peptide or pharmaceutically active fragments thereof. The term also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, cellular signal transduction. “Signal transduction” refers to the transmission of a molecular signal in the form of chemical modification by recruitment of protein complexes along a pathway that ultimately triggers a biochemical event in the cell. Signal transduction pathways are well known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduces a signaling response is “antagonistic” or “an antagonist.”

As used herein, the term “specific” or “specificity” can be used to refer to the ability of a molecule, e.g., a receptor, antibody, or an engager, to selectively bind to a target molecule, in contrast to non-specific or non-selective binding.

As used herein, the term “targeting modality” refers to a molecule, e.g., a polypeptide, that is genetically incorporated into a cell to promote antigen and/or epitope specificity that includes, but is not limited to, i) antigen specificity as it relates to a unique chimeric antigen receptor (CAR) or T cell receptor (TCR), ii) engager specificity as it relates to monoclonal antibodies or bispecific engagers, iii) targeting of transformed cells, iv) targeting of cancer stem cells, and v) other targeting strategies in the absence of a specific antigen or surface molecule.

“HLA deficient”, including HLA class I deficient, or HLA class II deficient, or both, refers to cells that either lack, or no longer maintain, or have reduced levels of surface expression of a complete MHC complex comprising an HLA class I protein heterodimer and/or an HLA class II heterodimer, such that the diminished or reduced level is less than the level naturally detectable by other cells or by synthetic methods. HLA class I deficiency can be achieved by functional deletion of any region of the HLA class I locus (chromosome 6p21), or deletion or reducing the expression level of HLA class I associated genes including, not being limited to, beta-2 microglobulin (B2M) gene, TAP 1 gene, TAP 2 gene and Tapasin. HLA class II deficiency can be achieved by functional deletion or reduction of HLA-II associated genes including, not being limited to, RFXANK, CIITA, RFX5 and RFXAP. It was previously unclear whether HLA complex deficient or altered iPSCs have the capacity to enter development, mature and generate functional differentiated cells while retaining modulated activity. In addition, it was previously unclear whether HLA complex deficient differentiated cells can be reprogrammed to iPSCs and maintained as pluripotent stem cells while having the HLA complex deficiency. Unanticipated failures during cellular reprogramming, maintenance of pluripotency and differentiation may be related to aspects including, but not limited to, development stage specific gene expression or lack thereof, requirements for HLA complex presentation, protein shedding of introduced surface expressing modalities, need for proper and efficient clonal reprogramming, and need for reconfiguration of differentiation protocols.

“Modified HLA deficient iPSC,” as used herein, refers to an HLA deficient iPSC that is further modified by introducing genes expressing proteins related, but not limited, to improved differentiation potential, antigen targeting, antigen presentation, antibody recognition, persistence, immune evasion, resistance to suppression, proliferation, co-stimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11b, NOTCH, RUNX1, IL15, 4-1BB, DAP10, DAP12, CD24, CD3z, 4-1BBL, CD47, CD113, and PDL1. The cells that are “modified HLA deficient” also include cells other than iPSCs.

The term “antibody” is used herein in the broadest sense and refers generally to an immune response generating molecule that contains at least one binding site that specifically binds to a target, wherein the target may be an antigen, or a receptor that is capable of interacting with certain antibodies. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term “antibody” includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, a single variable new antigen receptor (VNAR), a shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv) 2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells, T and B cells) and the signalling properties of each receptor. Fc-gamma receptors (FcγR) include several members, FcγRI (CD64), FcγRIIA (CD32), FcγRIIB (CD32), FcγRIIIA (CD16a), FcγRIIIB (CD16b), which differ in their antibody affinities due to their different molecular structures.

CD16, a FcγR receptor, has been identified to have two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16” (abbreviated as hnCD16), as used herein, refers to a natural or non-natural variant of CD16. The wildtype CD16 has low affinity and is subject to ectodomain shedding, a proteolytic cleavage process that regulates the cells surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO 2015/148926, the complete disclosure of which is incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

The term “adoptive cell therapy” as used herein refers to a cell-based immunotherapy that, as used herein, relates to the transfusion of autologous or allogeneic lymphocytes, such as CD34 cells, hemogenic endothelium cells, hematopoietic stem or progenitor cells, hematopoietic multipotent progenitor cells, T cell progenitor cells, NK cell progenitor cells, T cells, NKT cells, NK cells, B cells, or immune regulatory cells, genetically modified or not, that have been expanded ex vivo prior to said transfusion.

As used herein, the term “subject” refers to any animal, preferably a human patient, livestock, or other domesticated animal.

As used herein, the terms “treat,” “treatment” and the like, when used in reference to a subject in need of a therapeutic treatment, refer to obtaining a desired pharmacologic and/or physiologic effect, including without limitation achieving an improvement or elimination of the symptoms of a disease. The effect can be prophylactic in terms of completely or partially preventing a disease or symptom thereof and/or can be therapeutic in terms of achieving an improvement or elimination of symptoms, or providing a partial or complete cure for a disease and/or adverse effect attributable to the disease. The term “treatment” includes any treatment of a disease in a mammal, particularly in a human, and includes: (a) preventing the disease from occurring in a subject which can be predisposed to the disease but has not yet been diagnosed as having it; (b) inhibiting the disease, or arresting its development; (c) relieving the disease, or causing regression of the disease, or completely or partially eliminating symptoms of the disease; and/or (d) restoring the individual to a pre-disease state, such as reconstituting the hematopoietic system.

A “therapeutically sufficient amount”, as used herein, includes within its meaning a non-toxic, but sufficient and/or effective amount of a particular therapeutic agent and/or pharmaceutical composition to which it is referring to provide a desired therapeutic effect. The exact amount required will vary from subject to subject, depending on factors such as the patient's general health, the patient's age and the stage and severity of the condition being treated. In some embodiments, a “therapeutically sufficient amount” is sufficient and/or effective to ameliorate, reduce, and/or improve at least one symptom associated with a disease or condition of the subject being treated.

### A. Compositions and Methods for Production and Differentiation of Induced Pluripotent Stem Cells

In some aspects, the invention generally relates to a multistage process of differentiating naïve pluripotent cells to non-pluripotent cells or partially differentiated cells, including, mesoderm progenitor cells, mesodermal cells, definitive hemogenic endothelium, definitive hematopoietic stem or progenitor cells, CD34⁺ cells, multipotent progenitors (MPP) (capable of differentiating into myeloid, including neutrophil progenitors), T cell progenitors, NK cell progenitors; or fully differentiated terminal hematopoietic cells, such as, for example, T cells, B cells, NKT cells, or NK cells. In various embodiments, such naïve pluripotent cells may be obtained by reprogramming source non-pluripotent cells to induced pluripotent stem cells (iPSC), where the iPSCs retain one or more therapeutic attributes of the source cells. In some aspects, the invention relates to the compositions used in the disclosed methods; and cell populations, cell lines, clonal cells, or master cell banks generated using the disclosed methods.

Existing methods for culturing pluripotent cells, such as iPSCs, rely heavily on feeder cells or media pre-conditioned with feeder cells and containing fetal bovine serum; however, such environments may be unsuitable for producing cells for clinical and therapeutic use. For example, cells cultivated in such xeno-contaminated environments are generally considered unsuitable for human cell transplantation because the exposure to animal components may present a serious risk of immune rejection and transmitting unidentified pathogens to the treated patients, and could potentially reactivate animal retroviruses. Culture systems using animal-free and feeder-free culture media contemplated herein facilitate the manufacture of clinical-grade cell lines, particularly ESC, iPSC, and pluripotent stem cell derived T, B, NKT, or NK cell lines.

In some embodiments, the feeder-free environment is essentially free of human feeder cells and is not pre-conditioned by feeder cells, including without limitation, mouse embryonic fibroblasts, human fibroblasts, keratinocytes, and embryonic stem cells. In some embodiments, the feeder-free environment is further free of stromal cells, such as OP9 stromal cells. The feeder-free cell culture medium is suitable for use in culturing pluripotent cells, single-cell culture, dissociation, and passaging of pluripotent cells; cell sorting of pluripotent cells; generation of ground state pluripotent cells; maintenance of ground state pluripotency; induction of pluripotent cell differentiation; and maturation of effector cells from pluripotent cell differentiation.

In contrast to the methods used in the art, various aspects of the present invention avoid the formation of EB during differentiation. As provided in various embodiments, hematopoietic lineage cells derived from iPSCs are obtained by seeding clonal iPSC cells in a TGFβ free culture medium to maintain their ground or naïve state of pluripotency, differentiating the clonal iPSCs in a monolayer format without EB formation, and utilizing a step-wise strategy to apply a proper combination of small molecules, growth factors and/or cytokines in the early- and mid-stages of the differentiation. As such, aspects of the present invention enable direct transfer of expanded clonal iPSC to adherent culture in a form of monolayer for immediate differentiation without requiring formation of EB from iPSC.

The compositions provided herein are useful, in part, for the production of industrial- or clinical-grade pluripotent cells having reduced spontaneous differentiation as compared to cells generated or cultured in the absence of the compositions. In one embodiment, non-pluripotent cells are induced to become pluripotent cells and cultured to maintain pluripotency in long-term. In another embodiment, non-pluripotent cells are induced to become pluripotent cells and cultured to achieve and/or maintain reduced spontaneous differentiation as compared to cells cultured in the absence of the compositions. In another embodiment, non-pluripotent cells are induced to become pluripotent cells and cultured to achieve and/or maintain ground state pluripotency (see, for example, compositions in Tables 1 and 2).

In various embodiments, the compositions provided herein maintain ground state pluripotency, normal karyotypes, and genomic stability of one or more pluripotent cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more passages, including any intervening number of passages. In other embodiments, the compositions provided herein (see, for example, Table 2) maintain reduced spontaneous differentiation in one or more pluripotent cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more passages, including any intervening number of passages.

In various embodiments, the culture media provided herein may comprise any defined basal media suitable for supporting the maintenance and/or growth of stem cells, such as conventional human embryonic stem cell media. Examples of defined basal media which may be used in accordance with embodiments of the invention include, but are not limited to: Dulbecco's Modified Eagle Medium (“DMEM”), Basal Media Eagle (BME), DMEM/F-12 (1:1 DMEM and F-12 vol:vol); Medium 199; F-12 (Ham) Nutrient Mixture; F-10 (Ham) Nutrient Mixture; Minimal Essential Media (MEM), StemPro®-34, Williams' Media E; and RPMI 1640, all of which are available from Gibco-BRL/Life Technologies, Inc., Gaithersburg, Md., among others. Several versions of many of these media are available, which include, but are not limited to: DMEM 11966, DMEM 10314, MEM 11095, Williams' Media E 12251, Ham F12 11059, MEM-alpha 12561, and Medium-199 11151 (Gibco-BRL/Life Technologies). In various embodiments, the culture media may include, for example, one or more of the following: amino acids, vitamins, organic salts, inorganic salts, trace elements, buffering salts, sugars, ATP, and the like.

Small molecules, and classes thereof, for use in the cell culture media according to embodiments of the invention are described more fully below. In one embodiment, the composition comprises a cell culture medium and one or more of a TGFβ family protein, a Rho Kinase inhibitor (ROCKi), and a MEK inhibitor (MEKi) and WNT activator. In various embodiments, the composition does not comprise a TGFβ inhibitor (TGFβi). In various embodiments, one or more of the TGFβ family protein, ROCKi, MEKi and WNT activator may be added at one or multiple specific stages during iPSC generation, maintenance and/or differentiation for a predetermined duration. Such specific stages during iPSC generation (reprogramming) include, but are not limited to, somatic cell transfection (day 0), exogenous gene expression, increase of heterochromatin, loss of somatic cell identity, and iPSC colony formation. Specific stages during iPSC maintenance include, but are not limited to, single cell dissociation of iPSC colonies, single cell sorting of dissociated iPSCs, iPSC single cell clonal expansion, clonal iPSC master cell bank (MCB) cryopreservation, thawing of iPSC MCB, and optionally additional cryopreserve-thaw cycles of the iPSC MCB.

TABLE 1
Exemplary Media for iPSC Reprogramming and Maintenance

| Conventional hESC | Fate Reprogramming | Fate Maintenance |
|---|---|---|
| Medium (Conv.) | Medium (FRM) | Medium (FMM) |
| DMEM/F12 | DMEM/F12 | DMEM/F12 |
| Knockout Serum | Knockout Serum | Knockout Serum |
| Replacement (20%) | Replacement (20%) | Replacement (20%) |
| | N2 | |
| | B27 | |
| Glutamine | Glutamine | Glutamine (1x) |
| Non-Essential | Non-Essential Amino | Non-Essential Amino |
| Amino Acids (1x) | Acids (1x) | Acids (1x) |
| β-mercaptoethanol | β-mercaptoethanol | β-mercaptoethanol |
| (100 μM) | (100 μM) | (100 μM) |
| bFGF (0.2-50 | bFGF (2-500 | bFGF (2-500 |
| ng/mL) | ng/mL) | ng/mL) |
| | LIF (0.2-50 ng/mL) | LIF (0.2-50 ng/mL) |
| | Thiazovivin (0.1-25 μM) | Thiazovivin (0.1-25 μM) |
| | PD0325901 | PD0325901 |
| | (0.005-2 μM) | (0.005-2 μM) |
| | CHIR99021 (0.02-5 μM) | CHIR99021 (0.02-5 μM) |
| | SB431542 (0.04-10 μM) | |

| | |
|---|---|
| In combination with | Feeder-free, in combination with |
| MEF feeder cells | Matrigel ™ or Vitronectin |

TABLE 2
Exemplary Stage-Specific Media for
iPSC Reprogramming and Maintenance

| | | |
|---|---|---|
| | Fate | Fate |
| | Reprogramming | Maintenance |
| Stage of Processes | Medium (FRM2) | Medium (FMM2) |

Reprogramming

| | |
|---|---|
| Somatic Cell Transfec- | FRM (see Table 1) |
| tion (day 0) | without TGFβ |
| | inhibitor |
| Chromatin Restructuring | add HDAC inhibitor |
| (around day 2-3 post | (Valproic acid) |
| transfection) | |
| Loss of Somatic Cell | add TGFβ |
| Identity (around day | inhibitor |
| 6-8 post transfection) | |
| until iPSC colony | |
| formation (around | |
| day 13-15) | |

iPSC Maintenance

| | |
|---|---|
| iPSC colony | FMM (see Table 1), |
| dissociation | optionally with reduced |
| | concentrations of one or |
| | both of GSK3 inhibitor |
| | and MEK inhibitor, and |
| | optionally add Activin A |
| Single cell | FMM, optionally with |
| sorting | reduced concentrations of |
| | one or both of GSK3 |
| | inhibitor and MEK |
| | inhibitor and optionally |
| | add Activin A |
| Clonal expansion, | FMM, with reduced |
| banking, cryopreserve, | concentrations of one or |
| and thaw | both of GSK3 inhibitor |
| | and MEK inhibitor, and |
| | with Activin A |

Suitable nutrients/extracts may include, for example, KOSR (knockout serum replacement); L-glut; and NEAA (Non-Essential Amino Acids). Other medium additives may include, but are not limited to, MTG, ITS, BME, anti-oxidants (for example, ascorbic acid) and nicotinamide (NAM). In some embodiments, a culture medium of the present invention comprises one or more of the following cytokines or growth factors: epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), bone morphogenetic protein (BMP4), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon stem cells such as stem cell factor (SCF) and erythropoietin (EPO). These cytokines may be obtained commercially, for example from R&D Systems (Minneapolis, Minn.), and may be either natural or recombinant. In some other embodiments, the culture medium of the present invention comprises one or more of bone morphogenetic protein (BMP4), insulin-like growth factor-1 (IGF-1), basic fibroblast growth factor (bFGF), vascular endothelial growth factor (VEGF), hematopoietic growth factor (for example, SCF, GMCSF, GCSF, EPO, IL3, TPO, EPO), Fms-Related Tyrosine Kinase 3 Ligand (Flt3L); and one or more cytokines, for example, Leukemia inhibitory factor (LIF), IL3, IL6, IL7, IL11, and IL15. In some embodiments, the growth factors/mitogens and cytokines are stage and/or cell type specific in concentrations that are determined empirically or as guided by the established cytokine art. In some embodiments, the cytokines are added at specific stages of differentiation.

Any suitable vessel or cell culture container may be used as a support for cell cultures in the basal media and/or the cell culture supplements. In some embodiments, coating the surface of a culture vessel with adhesion-promoting matrices/substrata (for example, collagens, fibronectins, RGD-containing polypeptides, gelatins, and the like) however promotes attachment of the cells, and in some embodiments may enhance the effect of the cell culture medium and supplements disclosed herein and/or provide for feeder-free differentiation. Suitable substrates for culturing and passaging cells are known in the art and include, without limitation, vitronectin, gelatin, laminin, fibronectin, collagen, elastin, osteopontin, thrombospondin, mixtures of naturally occurring cell line-produced matrices such as Matrigel™, and synthetic or man-made surfaces such as polyamine monolayers and carboxy-terminated monolayers. In some embodiments, providing feeder-free conditions comprises culturing the cells on a matrix-coated surface/substrate. In some embodiments, the matrix is an extracellular matrix comprising a recombinant human fibronectin or fragment thereof and Fc-rhDLL4. In some embodiments, the recombinant human fibronectin is Retronectin®. In other embodiments, the matrix comprises Matrigel™ or vitronectin.

### ROCK Inhibitors

Rho associated kinases (ROCK) are serine/threonine kinases that serve downstream effectors of Rho kinases (of which three isoforms exist—RhoA, RhoB and RhoC). ROCK inhibitors suitable for use in compositions contemplated herein include, but are not limited to, polynucleotides, polypeptides, and small molecules. ROCK inhibitors (also referred to as “ROCKi”) contemplated herein may decrease ROCK expression and/or ROCK activity. Exemplary ROCK inhibitors include, but are not limited to, antibodies to ROCK, dominant negative ROCK variants, and siRNA and antisense nucleic acids that suppress expression of ROCK. Other exemplary ROCK inhibitors include, but are not limited to: thiazovivin, Y27632, Fasudil, AR122-86, Y27632 H-1152, Y-30141, Wf-536, HA-1077, hydroxyl-HA-1077, GSK269962A, SB-772077-B, N-(4-Pyridyl)-N′-(2,4,6-trichlorophenyl) urea, 3-(4-Pyridyl)-1H-indole, and (R)-(+)-trans-N-(4-Pyridyl)-4-(1-aminoethyl)-cyclohexanecarboxamide.

Exemplary ROCK inhibitors for use in the cell culture medium according to embodiments of the invention include thiazovivin, Y27632, pyrintegrin, Blebbistatin, and functional variants or derivatives thereof. In certain embodiments, the ROCK inhibitor is thiazovivin.

### ERK/MEK Inhibitors

Exemplary inhibitors of the ERK/MEK pathway suitable for use in compositions contemplated herein include, but are not limited to, antibodies to MEK or ERK, dominant negative MEK or ERK variants, and siRNA and antisense nucleic acids that suppress expression of MEK and/or ERK. Other exemplary ERK/MEK inhibitors (also referred to as “MEK inhibitors” or “MEKi”) include, but are not limited to, PD0325901, PD98059, UO126, SL327, ARRY-162, PD184161, PD184352, sunitinib, sorafenib, Vandetanib, pazopanib, Axitinib, GSKI 120212, ARRY-438162, RO5126766, XL518, AZD8330, RDEAI 19, AZD6244, FR180204, PTK787, and functional variants or fragments thereof.

Further illustrative examples of MEK/ERK inhibitors include the following compounds: 6-(4-Bromo-2-chloro-phenylamino)-7-fluoro-3-methyl-3H-benzoimidazol-e-5-carboxylic acid (2,3-dihydroxy-propoxy)-amide; 6-(4-Bromo-2-chloro-phenylamino)-7-fluoro-3-(tetrahydro-pyran-2-ylm-ethyl)-3H-benzoimidazole-5-carboxylic acid (2-hydroxy-ethoxy)-amide, 1-[6-(4-Bromo-2-chloro-phenylamino)-7-fluoro-3-methyl-3H-benzoimida-zol-5-yl]-2-hydroxy-ethanone, 6-(4-Bromo-2-chloro-phenylamino)-7-fluoro-3-methyl-3H-benzoimidazol-e-5-carboxylic acid (2-hydroxy-1,1-dimethyl-ethoxy)-amide, 6-(4-Bromo-2-chloro-phenylamino)-7-fluoro-3-(tetrahydro-furan-2-ylm-ethyl)-3H-benzoimidazole-5-carboxylic acid (2-hydroxy-ethoxy)-amide, 6-(4-Bromo-2-fluoro-phenylamino)-7-fluoro-3-methyl-3H-benzoimidazol-e-5-carboxylic acid (2-hydroxy-ethoxy)-amide, 6-(2,4-Dichloro-phenylamino)-7-fluoro-3-methyl-3H-benzoimidazole-5-carboxylic acid (2-hydroxy-ethoxy)-amide, 6-(4-Bromo-2-chloro-phenylamino)-7-fluoro-3-methyl-3H-benzoimidazol-e-5-carboxylic acid (2-hydroxy-ethoxy)-amide, referred to hereinafter as MEK inhibitor 1; 2-[(2-fluoro-4-iodophenyl)amino]-N-(2-hydroxyethoxy)-1,5-dimethyl-6-oxo-1,6-dihydropyridine-3-carboxamide; referred to hereinafter as MEK inhibitor 2; and 4-(4-bromo-2-fluorophenylamino)-N-(2-hydroxyethoxy)-1,5-dimethyl-6-oxo-1,6-dihydropyridazine-3-carboxamide or a pharmaceutically acceptable salt thereof.

In some embodiments, the MEK/ERK inhibitor is PD0325901.

### Wnt Activators

As used herein, the terms “Wnt signal-promoting agent,” “Wnt pathway activating agent,” “Wnt activator” or “Wnt pathway agonist,” refers to an agonist of the Wnt signaling pathway, including but not limited to an agonist of one or more of Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16. Wnt pathway agonists further include, but are not limited to, one or more of the following polypeptides or a fragment thereof: a Dkk polypeptide, a crescent polypeptide, a cerberus polypeptide, an axin polypeptide, a Frzb polypeptide, a T-cell factor polypeptide, or a dominant negative disheveled polypeptide.

Non-limiting examples of Wnt pathway agonists further include one or more of the following: a nucleic acid comprising a nucleotide sequence that encodes a Wnt polypeptide, a polypeptide comprising an amino acid sequence of a Wnt polypeptide, a nucleic acid comprising a nucleotide sequence that encodes an activated Wnt receptor, a polypeptide comprising an amino acid sequence of an activated Wnt receptor, a small organic molecule that promotes Wnt/β-catenin signaling, a small organic molecule that inhibits the expression or activity of a Wnt antagonist, an antisense oligonucleotide that inhibits expression of a Wnt antagonist, a ribozyme that inhibits expression of a Wnt antagonist, an RNAi construct, siRNA, or shRNA that inhibits expression of a Wnt antagonist, an antibody that binds to and inhibits the activity of a Wnt antagonist, a nucleic acid comprising a nucleotide sequence that encodes a β-catenin polypeptide, a polypeptide comprising an amino acid sequence of a β-catenin polypeptide, a nucleic acid comprising a nucleotide sequence that encodes a Lef-1 polypeptide, a polypeptide comprising an amino acid sequence of a Lef-1 polypeptide, and functional variants or fragments thereof.

### GSK-3β Inhibitors

GSK-3B inhibitors (also referred to as “GSK3 inhibitors” or “GSK3i”) are specific exemplary Wnt pathway agonists suitable for use in compositions contemplated herein, and may include, but are not limited to, antibodies that bind GSK-3β, dominant negative GSK-3β variants, and siRNA and antisense nucleic acids that target GSK-3β. Other exemplary GSK-3β inhibitors include, but are not limited to, Kenpaullone, 1-Azakenpaullone, CHIR99021, CHIR98014, AR-A014418, CT 99021, CT 20026, SB216763, AR-A014418, lithium, SB 415286, TDZD-8, BIO, BIO-Acetoxime, (5-Methyl-1H-pyrazol-3-yl)-(2-phenylquinazolin-4-yl)amine, Pyridocarbazole-cyclopenadienylruthenium complex, TDZD-8 4-Benzyl-2-methyl-1,2,4-thiadiazolidine-3,5-dione, 2-Thio(3-iodobenzyl)-5-(1-pyridyl)-[1,3,4]-oxadiazole, OTDZT, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione; TWSl 19 pyrrolopyrimidine compound, L803 H-KEAPPAPPQSpP-NH2 or its myristoylated form; 2-Chloro-1-(4,5-dibromo-thiophen-2-yl)-ethanone, SB216763, SB415286, and functional variants or fragments thereof. Exemplary GSK3 inhibitors for use in the cell culture media according to embodiments of the invention include CHIR99021, BIO, and Kenpaullone. In some embodiments, the GSK3 inhibitor is CHIR99021.

### TGFβ Receptor/ALK5 Inhibitors

TGFβ receptor (e.g., ALK5) inhibitors can include antibodies to, dominant negative variants of, and antisense nucleic acids that suppress expression of, TGFβ receptors (e.g., ALK5). Exemplary TGFβ receptor/ALK5 inhibitors (also referred to as “ALK5i”) include, but are not limited to, SB431542, A-83-01, 2-(3-(6-Methylpyridin-2-yl)-1H-pyrazol-4-yl)-1,5-naphthyridine, Wnt3a/BIO, BMP4, GW788388 (−{4-[3-(pyridin-2-yl)-1H-pyrazol-4-yl]pyridin-2-yl}-N-(tetrahydro-2H-pyran-4-yl)benzamide), SM16, IN-1130 (3-((5-(6-methylpyridin-2-yl)-4-(quinoxalin-6-yl)-1H-imidazol-2-yl)methyl)benzamide), GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride) and pyrimidine derivatives. Further, while “an ALK5 inhibitor” is not intended to encompass non-specific kinase inhibitors, an “ALK5 inhibitor” should be understood to encompass inhibitors that inhibit ALK4 and/or ALK7 in addition to ALK5, such as, for example, SB-431542. Without intending to limit the scope of the invention, it is believed that ALK5 inhibitors affect the mesenchymal to epithelial conversion/transition (MET) process. TGFβ/activin pathway is a driver for epithelial to mesenchymal transition (EMT). Therefore, inhibiting the TGFβ/activin pathway can facilitate MET (i.e., reprogramming) process.

It has been shown that inhibition of the TGFβ/activin pathway has similar effects of inhibiting ALK5. Thus, any inhibitor (e.g., upstream or downstream) of the TGFβ/activin pathway can be used in combination with, or instead of, ALK5 inhibitors as described in each paragraph herein. Exemplary TGFβ/activin pathway inhibitors include but are not limited to: TGFβ receptor inhibitors, inhibitors of SMAD 2/3 phosphorylation, inhibitors of the interaction of SMAD 2/3 and SMAD 4, and activators/agonists of SMAD 6 and SMAD 7. Furthermore, the categorizations described below are merely for organizational purposes and one of skill in the art would know that compounds can affect one or more points within a pathway, and thus compounds may function in more than one of the defined categories.

Specific examples of TGFβ receptor inhibitors include but are not limited to SU5416; 2-(5-benzo[1,3]dioxol-5-yl-2-tert-butyl-3H-imidazol-4-yl)-6-methylpyridine hydrochloride (SB-505124); lerdelimumb (CAT-152); metelimumab (CAT-192); GC-1008; ID11; AP-12009; AP-11014; LY550410; LY580276; LY364947; LY2109761; SB-505124; SB-431542; SD-208; SM16; NPC-30345; Ki26894; SB-203580; SD-093; Gleevec; 3,5,7,2′,4′-pentahydroxyflavone (Morin); activin-M108A; P144; soluble TBR2-Fc; and antisense transfected tumor cells that target TGFβ receptors.

Inhibitors of SMAD 2/3 phosphorylation can include antibodies to, dominant negative variants of and antisense nucleic acids that target SMAD2 or SMAD3. Specific examples of inhibitors include PD169316; SB203580; SB-431542; LY364947; A77-01; and 3,5,7,2′,4′-pentahydroxyflavone (Morin). Inhibitors of the interaction of SMAD 2/3 and SMAD4 can include antibodies to, dominant negative variants of and antisense nucleic acids that target SMAD2, SMAD3 and/or SMAD4. Specific examples of inhibitors of the interaction of SMAD 2/3 and SMAD4 include but are not limited to Trx-SARA, Trx-xFoxH1b and Trx-Lef1. Activators/agonists of SMAD 6 and SMAD 7 include but are not limited to antibodies to, dominant negative variants of and antisense nucleic acids that target SMAD 6 or SMAD 7.

### p38 MAPK Inhibitors

The p38 mitogen-activated protein kinase (MAPK) pathway plays a crucial role in the release of pro-inflammatory cytokines such as IL-6 and is stimulated by the inflammatory cytokine tumor necrosis factor-α (TNF-alpha), among other stressors. Four distinct subgroups within MAPKs have been identified including extracellular signal-regulated kinases (ERKs), c-jun N-terminal kinases (JNK/SAPK), ERK/Big MAP kinase 1 (BMK1) and the p38MAPK group of protein kinases. The p38 MAPK family includes p38α (MAPK14), p38β (MAPK11), p38γ (MAPK12), and p38δ (MAPK13). While the four p38 MAPK family members have different tissue expression patterns, it has been shown that p38 MAPK play critical roles in cellular responses, proliferation, survival, cell cycle, and migration in cancer, attracting the attention of p38 MAPK inhibitors for use in chemotherapy.

The various p38 inhibitors are structurally diverse small molecules that have a common mechanism of action that involves competitive inhibition of the adenosine-binding pocket (ATP-binding site) of p38. These inhibitors were selected for occupying the less-conserved surrounding hydrophobic areas of p38 binding site that induce conformational reorganization to block or reduce ATP binding to the p38 protein. Exemplary small molecule p38 MAPK inhibitors suitable for iPSC differentiation to definitive hemogenic endothelium (HE) cells and thus various derivative cells include, but are not limited to, cyclopropyl-{4-[4-(4-fluorophenyl)-2-piperidin-4-yl-thiazol-5-yl]pyrimidin-2-yl}amine (known as “DBM1285”) VX-745, VX-702, RO-4402257, SCIO-469, BIRB-796, SD-0006, PH-797804, AMG-548, LY2228820, SB-681323, GW-856553, RV568, CAS 219138-24-6, SB203580, and SB242235. DBM1285 was previously shown to inhibit TNF-α production. However, the present application discloses the use of p38 MAPK inhibitor for CD82 expression and the maintenance of the cell population expressing CD82, which subsequently increases the efficiency for obtaining definitive HE cells.

### AhR Inhibitors

The aryl hydrocarbon receptor (AhR) is a member of the Pern-Arnt-Sim (PAS) superfamily of transcription factors that are involved in sensing environmental signals such as changes in the circadian rhythm (BMAL1 and BMAL2), and oxygen tension or redox potential (HIF-1α, HIF-2α, HIF-3α), among others. AhR is expressed in hematopoietic stem and progenitor cells (HSPCs) and plays an important physiological role in hematopoiesis. Inhibition of AhR was used for expanding human umbilical cord blood-derived HSPCs. As demonstrated herein, however, AhR inhibition is useful for modulating PSC derived effector cell activation and function. Exemplary AhR inhibitors suitable for the disclosed use in the methods and compositions as described herein include, but are not limited to, CH-223191 (CAS 301326-22-7), UM729 (a pyrimidoindole derivative), UM171, and SR1 (StemRegenin 1).

### HDAC Inhibitors

Exemplary HDAC (histone deacetylase) inhibitors can include antibodies that bind to, dominant negative variants of, and siRNA and antisense nucleic acids that target HDAC. Histone acetylation is involved in histone and DNA methylation regulation. In general, at the global level, pluripotent cells have more histone acetylation, and differentiated cells have less histone acetylation. HDAC inhibitors facilitate activation of silenced pluripotency genes. Exemplary HDAC inhibitors suitable for use in compositions contemplated herein include, but are not limited to, TSA (trichostatin A), VPA (valproic acid), sodium butyrate (NaB), SAHA (suberoylanilide hydroxamic acid or vorinostat), sodium phenylbutyrate, depsipeptide (FR901228, FK228), trapoxin (TPX), cyclic hydroxamic acid-containing peptide 1 (CHAP1), MS-275, LBH589 and PXD101.

### Cytokines and Growth Factors

In some embodiments, the compositions and/or cell culture media provided herein are substantially free of cytokines and/or growth factors. In certain embodiments, the cell culture media contains one or more supplements including, but not limited to sera, extracts, growth factors, hormones, cytokines and the like, which may be added in a stage-specific manner to improve the quality and the efficiency of the reprogramming, maintenance and/or differentiation processes.

Various growth factors and their use in culture media are known and include, for example, ECM proteins, laminin 1, fibronectin, collagen IV isotypes, proteases, protease inhibitors, cell surface adhesion proteins, cell-signaling proteins, cadherins, chloride intracellular channel 1, transmembrane receptor PTK7, insulin-like growth factor, Inhibin beta A, inducers of the TGFβ/Activin/nodal signaling pathway, and Activin A. Cytokines used in the culture media may include, for example, one or more of the following: growth factors such as epidermal growth factor (EGF), acidic fibroblast growth factor (aFGF), basic fibroblast growth factor (bFGF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), leukemia inhibitory factor (LIF), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon stem cells such as stem cell factor (SCF) and erythropoietin (Epo).

In particular embodiments, the compositions and/or culture media may include a protein of the TGFβ family as the cytokine/growth factor component of the composition. Examples of TGFβ family proteins include, but are not limited to, Activin A, TGFβ, nodal and functional variants or fragments thereof. These cytokines/growth factors may be obtained commercially, and may be either natural or recombinant. Other cytokines, if used, may be added at concentrations that are determined empirically or as guided by the established cytokine art.

Reprogramming of Cells and iPSC Maintenance

Reprogramming factors known for stem cell reprogramming in the field could all be used with the present reprogramming methods. In one embodiment, the reprogramming factors include, but are not limited to, OCT4, SOX2, NANOG, KLF, LIN28, C-MYC, ECAT1, UTF1, ESRRB, SV40LT, HESRG, CDH1, TDGF1, DPPA4, DNMT3B, ZIC3, L1TD1, YAP1, and large T antigen (LTag), and any combinations thereof as disclosed in International Pub. Nos. WO 2015/134652 and WO 2017/066634, the disclosures of which are incorporated herein by reference. The reprogramming factors may also be in the form of polynucleotides encoding the reprogramming factors, and thus may be introduced to the non-pluripotent cells by vectors such as, a retrovirus, a Sendai virus, an adenovirus, an episome, a plasmid, and a mini-circle. In some embodiments, the one or more polynucleotides encoding at least one reprogramming factor are introduced by a lentiviral vector. In some embodiments, the one or more polynucleotides are introduced by an episomal vector. In various other embodiments, the one or more polynucleotides are introduced by a Sendai viral vector. In some embodiments, the one or more polynucleotides introduced by a combination of plasmids. See, for example, International Pub. No. WO 2019/075057A1, the disclosure of which is incorporated herein by reference.

Polynucleotides encoding these reprogramming factors may be comprised in a polycistronic construct (i.e., multiple coding sequences controlled by one promoter) or non-polycistronic construct (multiple coding sequences with some controlled by one promoter and some by a different promoter). The promoter may be, for example, CMV, EF1α, PGK, CAG, UBC, and other suitable promoters that are constitutive, inducible, endogenously regulated, or temporal-, tissue- or cell type-specific. In one embodiment, the promoter is CAG. In another embodiment, the promoter is EF1α. In some embodiments, the polycistronic construct may provide a single open reading frame (for example, multiple coding sequences are operatively linked by a self-cleaving peptide encoding sequence such as 2A) or multiple open reading frames (for example, multiple coding sequences linked by an Internal Ribosome Entry Site, or IRES).

An alternative method of obtaining iPSCs is to use a plasmid system that mediates short-lived transient and temporal transgene expression (see, e.g., “STTR system” in U.S. Application Pub. No. 20200270581 and “STTR2 system” as described in International Pub. No. WO 2022/072883, the relevant disclosure of each of which is incorporated herein by reference).

In some embodiments, the reprogramming of a non-pluripotent cell is initiated in the presence of a combination of small molecule compounds comprising a ROCK inhibitor, a MEK inhibitor, a WNT activator, an HDAC inhibitor and/or a TGFβ inhibitor, and iPSCs are generated after a sufficient period of time (see, e.g., International Pub. No. WO 2022/072883, the relevant disclosure of which is incorporated herein by reference).

The cells suitable for reprogramming generally include any non-pluripotent cells. Non-pluripotent cells include, but are not limited to, terminally differentiated cells; or multipotent or progenitor cells, which are not able to give rise to all three types of germ layer lineage cells. In some embodiments, the non-pluripotent cell for reprogramming is a primary cell, i.e., a cell isolated directly from human or animal tissue. In some embodiments, the non-pluripotent cell for reprogramming is a source specific cell, for example, donor-, disease-, or treatment response-specific. In some embodiments, the non-pluripotent cell for reprogramming is a primary immune cell. In some embodiments, the non-pluripotent cell for reprogramming is itself derived from a pluripotent cell, including an embryonic stem cell and/or an induced pluripotent stem cell. In some embodiments, the non-pluripotent cell for reprogramming is a derived immune effector cell, for example, an iPSC-derived non-natural or synthetic T- or NK-like cell.

In some other embodiments, the non-pluripotent cell for reprogramming is a genomically modified primary or derived cell. The genetic modification comprised in the non-pluripotent cell may include insertion, deletion or substitution in the genome, which leads to knock-in, knock-out or knock-down of a gene expression. The modified expression in the non-pluripotent cell for reprogramming may be constitutive or inducible (for example, development stage-, tissue-, cell-, or inducer-specific). In some embodiments, the insertion or substitution is a locus specific targeted integration. In some embodiments, the selected locus for integration is a safe harbor locus or an endogenous gene locus of interest.

In one embodiment, reprogramming of a genetically modified non-pluripotent cell is performed to obtain a genome-engineered iPSC comprising the same genetic modification(s). In some other embodiments, one or more such genomic edits may be introduced to the iPSC after reprogramming to obtain a genome-engineered iPSC. In some embodiments, the genome-engineered iPSC comprises a polynucleotide encoding a cytokine signaling complex comprising a partial or full peptide of a cell surface expressed exogenous cytokine and/or a receptor thereof, such that when subsequently differentiated toward a hematopoietic lineage cell through definitive hemogenic endothelium (HE), inclusion of the cytokine in the culture medium is not necessary or required. See, for example, International Application No. PCT/US2022/073396 and International Pub. Nos. WO 2022/098914 and WO 2022/098925, the entire disclosure of each of which is incorporated herein by reference. In one embodiment, the definitive HE cell comprises a genetic insertion of a polynucleotide encoding a cytokine signaling complex for IL15 signaling (IL 15 signaling complex), including but not limited to, IL15, IL 15RF (an IL15/IL 15R receptor fusion protein), or IL15A (an IL15/IL 15Ra fusion protein without an intracellular domain), as described in International Pub. Nos. WO 2019/191495 and WO 2019/126748, the entire disclosure of each of which is incorporated herein by reference. In various embodiments, the iPSC for genomic editing is a clonal line or a population of clonal iPS cells.

iPSC Differentiation
(1) iPSC to iHE

One aspect of the present invention provides methods and compostions for obtaining definitive hemogenic endothelium (HE) using pluripotent stem cells, such as induced pluripotent stem cells (iPSCs). As used herein, definitive hemogenic endothelium is a hemogenic cell population directed towards definitive hematopoiesis with the capacity to give rise to all hematopoietic cells including, but not limited to, pre-T cell progenitor cells, T cell progenitor cells, T cells, in addition to pre-NK cell progenitor cells, NK cell progenitor cells, NK cells, NKT cells, B cells, and other hematopoietic cells. In some aspects, the present invention provides compositions and methods for obtaining hematopoietic lineage cells from definitive HE cells, or from iPSC through definitive hemogenic endothelium differentiated from the iPSCs.

Generally, techniques for differentiating an iPSC or a definitive HE cell derived therefrom involve modulation of specific cellular pathways, either directly or indirectly, using polynucleotide-, polypeptide- and/or small molecule-based approaches. The developmental potency of a cell may be modulated, for example, by contacting a cell with one or more modulators. “Contacting”, as used herein, can involve culturing cells in the presence of one or more factors (such as, for example, small molecules, proteins, peptides, etc.). In some embodiments, a cell is contacted with one or more agents to induce cell differentiation. Such contact may occur for example, by introducing the one or more agents to the cell during in vitro culture. Thus, contact may occur by introducing the one or more agents to the cell in a cell culture medium. The cell may be maintained in the culture medium comprising one or more agents for a period sufficient for the cell to achieve the differentiation phenotype that is desired. In some other embodiments, “contact” occurs when one or more factors are introduced into the cell via vectors, as discussed below. In some embodiments, the one or more vectors are introduced by a retrovirus, Sendai virus, an adenovirus, an episome, mini-circle, vector system with expression cassette, or mRNA.

In various embodiments, the culture platform for generating hematopoietic cell lineages from iPSC or definitive hemogenic endothelium derived therefrom as provided herein do not comprise, or are essentially free of, an inhibitor of TGFβ/activin signaling pathways, including TGFβ receptor (TGFβR) inhibitors and ALK5 inhibitors. In some embodiments, the cell culture media for differentiating definitive hemogenic endothelium is free of stromal cells, such as OP9 stromal cells. In one embodiment, the culture platform comprises a seeding medium for maintaining a naïve iPSC. Achieving the ground or naïve state pluripotency of the iPSC is also important to obtain definitive HE cells and hematopoietic lineage cells by differentiating iPSC without forming EB intermediates. In addition, the efficiency of naïve iPSC differentiation into definitive HE is also greatly impacted by the use of monolayer culturing without forming EB and aggregates thereof. In some embodiments, the seeding medium comprises a ROCK inhibitor, and is free of, or essentially free of, TGFβR/ALK5 inhibitors. In some other embodiments, the seeding medium comprises a GSK3 inhibitor, but is free of TGFβR/ALK5 inhibitors. In yet some other embodiments, the seeding medium comprises a GSK3 inhibitor, a MEK inhibitor, and a Rho Kinase (ROCK) inhibitor.

One aspect of the present invention provides a culture medium for obtaining definitive hemogenic endothelium (HE) cells differentiated from pluripotent stem cells including iPSCs. In one embodiment, the culture medium comprises a BMP activator, bFGF, and optionally one or more of VEGF, a Wnt pathway activator, a p38 MAPK inhibitor, or any combination thereof. In one embodiment, the culture medium comprises a BMP activator, bFGF, VEGF and a Wnt pathway activator. In some other embodiments, the culture medium comprises a BMP activator, bFGF, VEGF, a Wnt pathway activator and a p38 MAPK inhibitor. In one embodiment, the VEGF, Wnt pathway activator, and/or p38 MAPK inhibitor is added to the medium comprising a BMP activator and bFGF after the differentiating cell from iPSCs obtain mesoderm progenitor specification. In one embodiment, the above culture medium is free of TGFβ receptor/ALK inhibitors. In one embodiment, the Wnt pathway activator is a GSK3B inhibitor. Without being limited to the theory, the small molecule p38 MAPK (mitogen-activated protein kinase) inhibitor contributes to improved maintenance of CD82 expression in HE cells as compared to without the inhibitor; whereas the BMP activator leads to a higher percentage of RUNX1-expressing cells in the HE population as compared to differentiation without the cytokine. In some embodiments, the BMP activator comprises BMP4. In some embodiments, the p38 MAPK inhibitor comprises at least one of DBM1285, VX-745, VX-702, RO-4402257, SCIO-469, BIRB-796, SD-0006, PH-797804, AMG-548, LY2228820, SB-681323, GW-856553, RV568, CAS 219138-24-6, SB203580, and SB242235. In one embodiment, the p38 MAPK inhibitor comprises DBM1285.

The pluripotent stem cells, optionally seeded and expanded as described above, are then differentiated to mesoderm progenitor cells, which expand in this stage. The expanded population of mesoderm progenitors is then differentiated to a mesodermal population with definitive hemogenic endothelium potential, and then into a population of definitive hemogenic endothelium (HE) cells. Alternatively, the method of generating iPSC-derived definitive hemogenic endothelium (iHE) could start from pluripotent stem cell-derived mesoderm progenitor cells, wherein the method comprises differentiating mesoderm progenitors to definitive hemogenic endothelium (iHE) cells. In some embodiments of the above methods, the iHE are amenable to cryopreservation. In some embodiments, the pluripotent stem cells are iPSCs. In some embodiments, the iPSCs are naïve iPSCs.

In some embodiments of the above methods for obtaining iPSC-derived hemogenic endothelium cells, the method comprises (i) differentiating pluripotent stem cells to obtain mesoderm progenitors or a population thereof by contacting the iPSCs with a medium comprising a BMP activator and bFGF, in addition to a basal medium; (ii) differentiating the mesoderm progenitors to obtain HE cells by contacting the mesoderm progenitors with a medium comprising a BMP activator, bFGF, VEGF, a Wnt pathway activator and optionally a p38 MAPK inhibitor, wherein the obtained HE cells comprise definitive HE cells.

In some embodiments of the above methods comprising (i) and (ii), the method further comprises (iii) sorting the obtained HE cells using antibodies that recognize cell surface markers comprising CD34, CD82, CD43, CD73, CXCR4, and/or CD93, thereby producing an enriched subpopulation of definitive HE cells. In some embodiments, the sorting uses an anti-CD34 antibody, and the enriched subpopulation of iHE cells are CD34⁺. In some embodiments, the sorting uses an anti-CD82 antibody, and the enriched subpopulation of definitive HE cells are CD82⁺. In some embodiments, the sorting uses both anti-CD34 and anti-CD82 antibodies, and the enriched subpopulation of definitive HE cells are CD34⁺CD82⁺. In some embodiments, the sorting uses anti-CD34, anti-CD82 and anti-CD43 antibodies, and the enriched subpopulation of definitive HE cells are CD34⁺CD82⁺CD43⁻. In some embodiments, the sorting uses CD34⁺, CD82⁺, CD43⁺, and CD73⁻. In some other embodiments, the sorting uses CD34⁺, CD82⁺, CD43⁺, CD73⁺, and CXCR4⁻. In some embodiments, the sorting uses CD34⁺, CD82⁺, and CD93⁻. In some embodiments, the sorting uses CD34⁺, CD82⁺, CD43⁻, and CD93⁻. In some embodiments, the sorting uses CD34⁺, CD43⁺, and CD73⁻. In some other embodiments, the sorting uses CD34⁺, CD43⁺, CD73⁺, and CXCR4⁻.

(2) iPSC, or iHE, to T Lineage Cells

Provided herein is a culture platform for generating T cell progenitors or T cells from definitive hemogenic endothelium. In one embodiment, the culture medium comprises SCF, Flt3L, IL7, and optionally a ROCK inhibitor, TPO, and IL3, wherein the medium is free of one or more of VEGF, bFGF, and BMP activators. In some embodiments, the medium for differentiating definitive HE into a T cell progenitor comprises a ROCK inhibitor, SCF, Flt3L, TPO, and IL7; and is without a BMP activator. In some embodiments, the medium for differentiating T cell progenitors to T cells comprises SCF, Flt3L, and IL7; and is without TPO, IL3, BMP activators, or ROCK inhibitors. In some embodiments, the ROCK inhibitor is thiazovivin or Y27632. In some embodiments the ROCK inhibitor is Y27632. In some embodiments, the BMP activator is BMP4. In some embodiments, Notch factors are used in the culture platform for generating a T cell progenitor or T cell from iPSC or definitive HE cells. In some embodiments, Notch factors, including Jag1, Jag2, DLL-1, DLL-3 and DLL-4, can be introduced as soluble peptides, peptides conjugated to beads, peptides conjugated to a culture surface, peptides comprised in an extracellular matrix coated on a culture surface, or peptides presented by stromal cells.

Further provided is a method of using a multistage process to generate pluripotent stem cell-derived T cell progenitors or T cells. Generally, the method begins with seeding and optionally expanding pluripotent stem cells, which are differentiated to mesoderm progenitors and subsequently to HE cells, and the HE cells may be optionally sorted to obtain enriched definitive HEs for subsequent T lineage cell differentiation. Alternatively, the HEs could be used as starting cells for T lineage cell differentiation. The methods and various embodiments for differentiating iPSCs to HE cells, including the methods of sorting HE cells are as described above, and methods of differentiating HE cells to T lineage cells subsequent to, or independent of, iPSC to HE cell differentiation are provided herein.

In some embodiments of the method for differentiating the HE cells to T cell progenitors or T cells, the method comprises contacting HE cells with a medium comprising one or more of the growth factors and cytokines comprising SCF, Flt3L, and IL7, and optionally, one or more factors comprising TPO, IL3, and a ROCK inhibitor; wherein the medium is free, or essentially free, of one or more of VEGF, bFGF, and a BMP activator. In some embodiments, the pluripotent stem cells are iPSCs. In some embodiments of the above methods, differentiating the iHE cells to pre-iproT comprises contacting the iHE cells with a medium comprising a ROCK inhibitor, SCF, Flt3L, TPO, and IL7. In other embodiments, differentiating the pre-iproT to ipro-T or iT comprises contacting the pre-iproT cells with a medium comprising SCF, Flt3L, and IL7, wherein the medium is free, or essentially free, of one or more of VEGF, bFGF, a BMP activator, and a ROCK inhibitor. In some embodiments of the above methods, Notch factors are used in the culture media for generating a T cell progenitor or T cell. In some embodiments, Notch factors, including Jag1, Jag2, DLL-1, DLL-3 and DLL-4, can be introduced as soluble peptides, peptides conjugated to beads, peptides conjugated to the surface, peptides comprised in an extracellular matrix coated on a cell culture surface, or peptides presented by cells. In some embodiments, differentiating the iHE cells to the T lineage cells occurs in the absence of OP9 stromal cells (OP9-free hematopoeitic cell differentiation). In some embodiments, the OP9-free differentiation of iHE is in the presence of an extracellular matrix comprising a recombinant human fibronectin or fragment thereof and Fc-rhDLL4. In some embodiments, the recombinant human fibronectin is Retronectin®.

(3) iPSC, or iHE, to NK Lineage Cells

NK lineage cells could be differentiated either from iPSC through iPSC derived definitive hemogenic endothelium (HE) cells, or directly from definitive HE cells.

Compositions and methods for differentiating iPSC to definitive HE cells including HE cell sorting, and various embodiments thereof are as described in the previous sections. Here, additional culture platforms and methods for differentiating definitive hemogenic endothelium cells to obtain NK lineage cells, including NK progenitors and NK cells, are further provided.

In some embodiments of the culture platform for obtaining NK lineage cells from HE cells, the culture platform comprises at least a medium comprising SCF, Flt3L, IL7, IL3, and optionally one or more of IL15, IL21, TPO, a ROCK inhibitor, an AhR antagonist, a 4-1BB agonist, and nicotinamide, wherein the medium is free of one or more of VEGF, bFGF, BMP activators, OP9 stromal cells, and feeder cells. In some embodiments of the medium for generating NK progenitors or NK cells, the medium is free of K562 feeder cells or engineered variants thereof. In some embodiments, the ROCK inhibitor comprises thiazovivin or Y27632.

In some embodiments of the medium for generating NK progenitors or NK cells, the medium comprises an AhR antagonist. In some embodiments, the AhR antagonist is a small molecule AhR inhibitor. Without being limited by theory, in some embodiments, the AhR inhibitor in the medium is useful for NK cell progenitor or NK cell expansion and/or activation. In some embodiment the AhR inhibitor comprises at least one of CH-223191, UM729, UM171, and SR1. In some embodiments, the medium for generating NK progenitors or NK cells comprises CH-223191. In some embodiments, in addition to one or more of an AhR antagonist, NK expansion, maturation, and/or activation is further in the presence of nicotinamide (NAM) comprised in the medium for generating NK progenitors or NK cells.

Further provided is a method of using a multistage process to generate pluripotent stem cell-derived NK progenitors or NK cells. Generally, the method begins with seeding and optionally expanding pluripotent stem cells, which are differentiated to mesoderm progenitors and subsequently to HE cells, and the HE cells may be optionally sorted to obtain enriched definitive HEs for subsequent NK lineage cell differentiation. Alternatively, the HEs could be used as starting cells for NK lineage cell differentiation. The methods and various embodiments for differentiating iPSCs to HE cells, including the methods of sorting HE cells are as described above, and methods of differentiating HE cells to NK lineage cells subsequent to, or independent of, iPSC to HE cell differentiation are provided herein.

In some embodiments, the method of differentiating HE cells to NK lineage cells comprises contacting the iHE cells with a composition comprising SCF, Flt3L, IL3, and IL7; and optionally one or more of a ROCK inhibitor, TPO, IL 15, immobilized IL21, a 4-1BB agonist, an AhR antagonist, and nicotinamide, thereby obtaining NK lineage cells, including NK progenitors and NK cells. In some embodiments of the method, the IL15 is comprised in the culture medium. In some other embodiments of the method, the IL15 is in the form of an IL15 signaling complex encoded by an exogenous polynucleotide that is introduced to the definitive HE cells by genetic insertion, wherein the IL15 signaling complex comprises a partial or full peptide of cell surface expressed exogenous IL 15 and/or a receptor thereof. In some embodiments, differentiating the iHE cells to NK progenitors or NK cells occurs in the absence of OP9 stromal cells. In some embodiments, differentiating the iHE cells to NK progenitors or NK cells occurs in the presence of an extracellular matrix comprising a recombinant human fibronectin or fragment thereof and Fc-rhDLL4, such that OP9 stromal cells are not needed. In some embodiments, the recombinant human fibronectin is Retronectin®. In some embodiments, the method of differentiating the iHE cells to iNK cells comprises contacting the iHE cells with a composition comprising SCF, Flt3L, IL3, IL7, a ROCK inhibitor, immobilized IL21, a 4-1BB agonist and optionally one or both of TPO and IL15, thereby obtaining iPSC-derived NK progenitors or NK cells.

In some embodiments, the method of differentiating the iHE cells to iNK cells further comprises contacting the iPSC-derived NK progenitors or NK cells with one or both of an AhR antagonist and nicotinamide, thereby expanding and/or activating the iPSC-derived NK cells. In some embodiments, NK activation occurs in the presence of a small molecule AhR inhibitor, thereby modulating the NK lineage cell maturation.

### B. Cell Populations and Cell Lines Generated From the Methods and Compositions Provided Herein

In light of the above, one of the advantages offered by the methods described herein is the enhanced viability and survival of culturing, passaging, and dissociating single pluripotent cells without EB formation for pluripotent stem cell differentiation. Dissociation of cells into single cells, such as into a single cell suspension, can be accomplished by enzymatic or mechanical means. Any enzymatic agent known in the art to allow dissociation of cells into single cells may be used in embodiments of the methods herein. In one embodiment, the dissociation agent is selected from Trypsin/EDTA, TrypLE-Select, Collagenase IV and Dispase. A chelator, such as EDTA, Accutase, or AccuMax, may also be used, alone or in combination with an enzymatic agent, in dissociating cells in accordance with the methods contemplated herein. The dissociation agent may be dissolved in calcium- and magnesium-free PBS to facilitate dissociation to single cells. To enhance the survival of the cells during and after dissociation, in some embodiments, a survival promoting substance is added, for example, one or more growth factors, inhibitors of cellular pathways involved in cell death and apoptosis, or conditioned media. In one embodiment, the survival promoting substance is a ROCK inhibitor, including but not limited to thiazovivin.

In some embodiments, the pluripotent stem cells are iPSCs. In some embodiments, the iPSCs are naïve iPSCs. In some embodiments, the iPSCs are reprogrammed from immune cells of a specific donor or patient. In some embodiments, the cells cultured after reprogramming are induced to differentiate for at least 1, 2, 3, 4, 5, 6, 7, 8, 9, 10, 11, 12, 15, 18, 20, 22, 24, 26, 28, 30, 32, 35, 40, 42, or 45 days, or any number of days in between. In some embodiments, the cells cultured after reprogramming are induced for about 1 to 42 days, 2 to 40 days, 2 to 35 days, 2 to 20 days, 2 to 10 days, 4 to 30 days, about 4 to 24 days, about 6 to 22 days, or about 8 to about 12 days.

In some embodiments, the iPSCs are genomically engineered. In some embodiments, the iPSC for differentiation comprises one or more genetic imprints. In some embodiments, the genetic imprints of the pluripotent stem cells comprise (i) one or more genetically modified modalities obtained through genomic insertion, deletion or substitution in the genome of the pluripotent cells during or after reprogramming a non-pluripotent cell to iPSC; or (ii) one or more retainable therapeutic attributes of a source specific immune cell that is donor-, disease-, or treatment response-specific, and wherein the pluripotent cells are reprogrammed from the source specific immune cell, wherein the iPSC retain the source therapeutic attributes, which are also comprised in the iPSC-derived hematopoietic lineage cells. In some embodiments, the genetically modified modalities comprise one or more of: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells thereof. In some other embodiments, the genetically modified modalities comprise one or more of (i) deletion, disruption, or reduced expression of B2M, TAP1, TAP2, Tapasin, NLRC5, PD1, LAG3, TIM3, RFXANK, CITTA, RFX5, or RFXAP; or (ii) introduction or increased expression of HLA-E, HLA-G, hnCD16, 4-1BBL, CD3, CD4, CD8, CD47, CD137, CD80, PDL1, A_{2A}R, CAR, TCR, or a surface triggering receptor for bi- or multi-specific engagers. In some embodiments, the surface triggering receptor is universal, i.e., compatible with any effector cell type, and the effector cells expressing the universal surface triggering receptor can couple with the same bi- or multi-specific engager irrespective of its cell type. In still some other embodiments, the hematopoietic lineage cells comprise the therapeutic attributes of the source specific immune cell relating to one or more of (i) antigen targeting receptor expression; (ii) HLA presentation or lack thereof; (iii) resistance to tumor microenvironment; (iv) induction of bystander immune cells and immune modulations; (v) improved on-target specificity with reduced off-tumor effect; (vi) resistance to treatment such as chemotherapy; and (vii) improved homing, persistence, and cytotoxicity.

In some embodiments, the engager is cell type specific, i.e., the engager binds to and/or activates a particular immune cell type. In particular embodiments, the engager is cell type independent, i.e., the engager binds to and/or activates multiple immune cells, e.g., T cells, NK cells, NKT cells, B cells, macrophages, or neutrophils.

In some embodiments, the iPSCs comprise one or more targeted edits at one or more desired sites, wherein the one or more targeted edits remain intact and functional in expanded iPSCs or iPSC-derived non-pluripotent cells at the respective selected editing site. The targeted editing introduces into the genome of the iPSC, and derivative cells therefrom, insertions, deletions, and/or substitutions (i.e., targeted integration and/or in/dels at selected sites). In some embodiments, the iPSC and its derivative hematopoietic cells comprise one or more of B2M negative, HLA-E/G, PDL1, A2AR, CD47, LAG3 negative, TIM3 negative, TAP1 negative, TAP2 negative, Tapasin negative, NLRC5 negative, PD1 negative, RFKANK negative, CITTA negative, RFX5 negative, and RFXAP negative. These cells with modified HLA class I and/or II have increased resistance to immune detection, and therefore present improved in vivo persistence. Moreover, such cells can avoid the need for HLA matching in adoptive cell therapy and thus provide a source of universal, off-the-shelf therapeutic regimens.

In some embodiments, the iPSC and its derivative hematopoietic cells comprise one or more of hnCD16, 4-1BBL, CD3, CD4, CD8, CAR, TCR, CD137 or CD80. Such cells have improved immune effector ability.

In some embodiments, the iPSC and its derivative hematopoietic cells comprise a surface triggering receptor for coupling with bi- or multi-specific engagers. Such cells have improved tumor targeting specificity. In some embodiments, the iPSC and its derivative hematopoietic cells are antigen specific. In some embodiments, the iPSC and its derivative hematopoietic cells comprise a polynucleotide encoding a cytokine signaling complex comprising a partial or full peptide of a cell surface expressed exogenous cytokine and/or a receptor thereof, such that inclusion of the cytokine in a culture medium is not necessary or required.

As described above, the methods include strategies for enriching a population of cells with specific characterizations at various stages of the methods. In one embodiment, the method of enriching pluripotent stem cells from a cell population comprises making a single cell suspension by dissociating the cells in the population and resuspending the cells. The dissociated cells may be resuspended in any suitable solution or media for maintaining cells or performing cell sorting. In various embodiments, enrichment provides a method for deriving clonal iPSC colonies in a relatively short time, thereby improving the efficiency of iPSC generation. Enrichment may comprise sorting a population of cells by identifying and obtaining cells expressing markers of pluripotency, thereby obtaining a population of enriched pluripotent cells. An additional enrichment methodology comprises the depletion of cells expressing markers of differentiation, non-reprogrammed or non-pluripotent cells. In some embodiments, the cells for sorting are pluripotent cells. In some embodiments, the cells for sorting are reprogramming cells. In some embodiments, the cells for sorting have been induced to reprogram for at least 1, 2, 3, 4, 5, 6, 7, 8 or more days, but no more than 25, 26, 28, 30, 32, 35, 40 days, or any number of days in between.

Cells may be sorted by any suitable method of sorting cells, such as by magnetic bead or flow cytometry (FACS) sorting. iPSCs may be sorted based on one or more markers of pluripotency, including without limitation, expression of SSEA3/4, TRA1-60/81, TRA1-85, TRA2-54, GCTM-2, TG343, TG30, CD9, CD29, CD133/prominin, CD140a, CD56, CD73, CD105, OCT4, NANOG, SOX2, KLF4, SSEA1 (Mouse), CD30, SSEA5, CD90 and/or CD50. In various embodiments, iPSCs are sorted based on at least two, at least three, or at least four markers of pluripotency. In certain embodiments, iPSCs are sorted based on expression of SSEA4, and in some embodiments based on expression of SSEA4 in combination with TRA1-81 and/or TRA1-60. In certain embodiments, iPSCs are sorted based on SSEA4, TRA1-81, or TRA1-60, and/or CD30 expression. In one embodiment, iPSCs are sorted based on SSEA4, TRA1-81 and CD30. In another embodiment, iPSCs are sorted based on SSEA4, TRA1-60 and CD30. In some embodiments, cells are initially depleted for non-reprogrammed cells using one or more surface markers of differentiating cells including, but not limited to, CD13, CD26, CD34, CD45, CD31, CD46 and CD7, and then enriched for pluripotent markers such as SSEA4, TRA1-81 and/or CD30.

In one embodiment, enrichment provides a method for obtaining clonal pluripotent stem cell-derived differentiating cell colonies in a relatively short time, thereby improving the efficiency of generating pluripotent stem cell-derived differentiated cells at various stages. In one embodiment, enrichment provides a method for deriving a population of CD34 expressing HE cells, a population of CD34 expressing HSC cells, a population of iHE cells, a population of T or NK progenitors, and/or a population of T or NK cells, thereby improving the efficiency of generating each of the cell populations. Enrichment may comprise sorting a population of cells, to identify and obtain cells expressing specific characteristic marker(s) indicative of differentiation stage/cell types. As discussed above, one or more antibodies that recognize one or more cell surface markers comprising CD34, CD82, CD43, CD73, CXCR4, and/or CD93 may be used to produce an enriched subpopulation of iHE cells. In some embodiments, the antibodies for cell sorting comprise an anti-CD34 antibody, and the enriched subpopulation of iHE cells are CD34⁺. In some embodiments, the antibodies for cell sorting comprise an anti-CD82 antibody, and the enriched subpopulation of iHE cells are CD82⁺. In some embodiments, the antibodies for sorting comprise both an anti-CD34 antibody and an anti-CD82 antibody, and the enriched subpopulation of iHE cells are CD34⁺CD82⁺. In some embodiments, the antibodies for cell sorting comprise an anti-CD34, an anti-CD82 and an anti-CD43 antibody, and the enriched subpopulation of iHE cells are CD34⁺CD82 CD43⁻. An additional enrichment methodology comprises the depletion of cells expressing markers representing undesired cell types to obtain an enriched population of desired cell types.

As such, another aspect of the invention provides a composition comprising one or more cell populations, cell lines, or clonal cells of (i) pluripotent stem cell-derived CD34 HE cells (iCD34), wherein the iCD34 cells have capacity to differentiate into multipotent progenitor cells, and wherein the iCD34 cells are CD34⁺CD43⁻; (ii) pluripotent stem cell-derived definitive hemogenic endothelium (iHE), wherein the iHE cell line or clonal cells are CD82⁺, or CD82⁺ and at least one of CD34⁺, CD93⁺, CXCR4⁺, CD73⁺, and CXCR4 CD73⁺; (iii) pluripotent stem cell-derived multipotent progenitor cells (iMPP), wherein the iMPP cells are CD34⁺CD45⁺; (iv) pluripotent stem cell-derived T cell progenitors (ipro-T), wherein the T cell progenitors are CD34⁺CD45⁺CD7⁺; (v) pluripotent stem cell-derived T cells (iT), wherein the T cells are CD45⁺CD4⁺CD3⁺ or CD45⁺CD8⁺CD3⁺; (vi) pluripotent stem cell-derived NK progenitors (ipro-NK), wherein the NK progenitors are CD45⁺CD56⁺CD7⁺CD3⁺; and (vii) pluripotent stem cell-derived NK cells (iNK), wherein the NK cells are CD45⁺CD56⁺NKp46⁺. In some embodiments, the above compositions, cell populations, cell lines or clonal cells are amenable to cryopreservation. In some embodiments, the compositions, cell populations, cell lines or clonal cells are amenable to ambient storage conditions for more than 12 hrs, 24 hrs, 36 hrs, 48 hrs, but not longer than 3 days, 4 days, 5 days, 6 days, or a week.

C. Therapeutic Use of iPSC Derived Immune Cells

In one aspect, the present invention also provides a composition comprising an isolated population or subpopulation of immune cells that have been derived from iPSC using the methods and compositions as disclosed, wherein the immune cells are suitable for cell based adoptive therapies. In one embodiment, the isolated population or subpopulation of immune cells comprises iPSC-derived HSC cells. In one embodiment, the isolated population or subpopulation of immune cells comprises iPSC-derived T cells. In one embodiment, the isolated population or subpopulation of immune cells comprises iPSC-derived NK cells. In some embodiments, an isolated population or subpopulation of immune cells that have been derived from iPSC comprises an increased number or ratio of naïve T cells, stem cell memory T cells, and/or central memory T cells. In one embodiment, the isolated population or subpopulation of immune cells that has been derived from iPSC comprises an increased number or ratio of type I NKT cells. In another embodiment, the isolated population or subpopulation of immune cells that has been derived from iPSC comprises an increased number or ratio of adaptive NK cells. In some embodiments, the isolated population or subpopulation of HSC cells, T cells, or NK cells derived from iPSC are allogenic. In some other embodiments, the isolated population or subpopulation of HSC cells, T cells, or NK cells derived from iPSC are autogenic.

A variety of diseases may be ameliorated by introducing immune cells according to aspects of the invention to a subject suitable for adoptive cell therapy. Examples of diseases including various autoimmune disorders, including but not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barré syndrome, idiopathic thrombocytopenia purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's); hematological malignancies, including but not limited to, acute and chronic leukemias, lymphomas, multiple myeloma and myelodysplastic syndromes; solid tumors, including but not limited to, tumor of the brain, prostate, breast, lung, colon, uterus, skin, liver, bone, pancreas, ovary, testes, bladder, kidney, head, neck, stomach, cervix, rectum, larynx, or esophagus; and infections, including but not limited to, HIV-(human immunodeficiency virus), RSV- (Respiratory Syncytial Virus), EBV- (Epstein-Barr virus), CMV- (cytomegalovirus), adenovirus- and BK polyomavirus-associated disorders.

Particular embodiments of the present invention are directed to methods of treating a subject in need thereof by administering to the subject a composition comprising any of the cells described herein. In some embodiments, the composition comprising any of the cells described herein may be administered in combination with a therapeutic agent. The therapeutic agent and/or composition may be administered before, during or after the onset of disease or injury. The treatment of ongoing disease, where the treatment stabilizes or reduces the undesirable clinical symptoms of the patient, is of particular interest.

In some embodiments, the subject has a disease, condition, and/or an injury that can be treated, ameliorated, and/or improved by a cell therapy. Some embodiments contemplate that a subject in need of cell therapy is a subject with an injury, disease, or condition, whereby a cell therapy, e.g., a therapy in which a cellular material is administered to the subject, can treat, ameliorate, improve, and/or reduce the severity of at least one symptom associated with the injury, disease, or condition. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g. a hyperproliferative disorder or a cancer of the hematopoietic system, a subject having or at risk of developing a tumor, e.g., a solid tumor, a subject who has or is at risk of having a viral infection or a disease associated with a viral infection.

Accordingly, aspects of the present invention further provide pharmaceutical compositions comprising the pluripotent cell derived hematopoietic lineage cells made by the methods and compositions disclosed herein, wherein the pharmaceutical compositions further comprise a pharmaceutically acceptable medium. In one embodiment, the pharmaceutical composition comprises the pluripotent cell derived T cells made by the methods and composition disclosed herein. In one embodiment, the pharmaceutical composition comprises the pluripotent cell derived NK cells made by the methods and composition disclosed herein.

Additionally, aspects of the present invention provide therapeutic use of the above pharmaceutical compositions by introducing/administering the composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus.

As a person of ordinary skill in the art would understand, both autologous and allogeneic immune cells, including hematopoietic lineage cells derived from iPSC based on the methods and compositions provided herein, can be used in cell therapies. Autologous cell therapies can have reduced infection, low probability for GvHD, and rapid immune reconstitution. Allogeneic cell therapies can have an immune-mediated graft-versus-malignancy (GVM) effect, and a low rate of relapse. For autologous transplantation, in some embodiments, the isolated population of derived hematopoietic lineage cells are either a complete or partial HLA-match with the patient. In other embodiments, the derived hematopoietic lineage cells are not HLA-matched to the subject, wherein the derived hematopoietic lineage cells are NK cells or T cells with HLA-I and/or HLA-II deficiency. Based on the specific conditions of the patient or subject in need of the cell therapy, a person of ordinary skill in the art would be able to determine which specific type of therapy to administer.

In some embodiments, the number of derived hematopoietic lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of derived hematopoietic lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to about 1.67×10⁶ cells/kg for a 60 kg patient.

In one embodiment, the number of derived hematopoietic lineage cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In some embodiments, the therapeutic use of derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every 25 days, every 30 days, every 35 days, every 40 days, every 45 days, or every 50 days, or any number of days in-between. In some embodiments, the multi-dose treatment comprises three, four, or five, once-weekly doses. In some embodiments of the multi-dose treatment comprising three, four, or five, once-weekly doses further comprise an observation period for determining whether additional single or multi doses are needed.

Some variation in dosage, frequency, and protocol will necessarily occur depending on the condition of the subject being treated. The person responsible for administration will, in any event, determine the appropriate dose, frequency and protocol for the individual subject.

The compositions comprising a population of derived hematopoietic lineage cells of embodiments of the invention can be sterile, and can be suitable and ready for administration (i.e., can be administered without any further processing) to human patients/subjects. A cell-based composition that is ready for administration means that the composition does not require any further processing or manipulation prior to transplant or administration to a subject. In other embodiments, the invention provides an isolated population of derived hematopoietic lineage cells that are expanded and/or modulated prior to administration with one or more agents including small chemical molecules. The compositions and methods for modulating immune cells including iPSC-derived effector cells are described in greater detail, for example, in International Pub. No. WO 2017/127755, the relevant disclosure of which is incorporated herein by reference. For derived hematopoietic lineage cells that are genetically engineered to express recombinant TCR or CAR, the cells can be activated and expanded using methods as described, for example, in U.S. Pat. No. 6,352,694. In certain embodiments, the primary stimulatory signal and the co-stimulatory signal for the derived hematopoietic lineage cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent is in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Pub. Nos. 2004/0101519 and 2006/0034810 for artificial antigen presenting cells (aAPCs) that are contemplated for use in activating and expanding T lymphocytes, in accordance with the present disclosure.

The sterile, therapeutically acceptable compositions suitable for administration to a patient can include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of the present disclosure (see, e.g., Remington's Pharmaceutical Sciences, 17^{th} ed. 1985, the disclosure of which is hereby incorporated by reference in its entirety).

These pharmaceutically acceptable carriers and/or diluents can be present in amounts sufficient to maintain a pH of the therapeutic composition of between about 3 and about 10. As such, the buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in some embodiments, the use of a pharmaceutically acceptable cell culture medium in particular compositions and/or cultures disclosed herein. Such compositions are suitable for administration to human subjects. Generally speaking, any medium that supports the maintenance, growth, and/or health of the iPSC-derived effector cells in accordance with embodiments of the invention are suitable for use as a pharmaceutical cell culture medium. In particular embodiments, the pharmaceutically acceptable cell culture medium is a serum free and/or feeder-free medium. In various embodiments, the serum-free medium is animal-free, and can optionally be protein-free. Optionally, the medium can contain biopharmaceutically acceptable recombinant proteins. An “animal-free” medium refers to a medium wherein the components are derived from non-animal sources. Recombinant proteins replace native animal proteins in animal-free medium and the nutrients are obtained from synthetic, plant or microbial sources. A “protein-free” medium, in contrast, is defined as being substantially free of protein. One having ordinary skill in the art would appreciate that the above examples of media are illustrative and in no way limit the formulation of media suitable for use in the present invention and that there are many suitable media known and available to those in the art.

### EXAMPLES

The following examples are offered by way of illustration and not by way of limitation.

### Example 1—Materials and Methods

hiPSC Maintenance in Small Molecule Culture:

hiPSCs were routinely passaged as single cells once confluency of the culture reached 75%-90%. For single-cell dissociation, hiPSCs were washed once with PBS (Mediatech) and treated with Accutase (Millipore) for 3-5 min at 37° C. followed with pipetting to ensure single-cell dissociation. The single-cell suspension was then mixed in equal volume with conventional medium, centrifuged at 225×g for 4 min, resuspended in FMM, and plated on a Matrigel-coated surface. Passages were typically 1:6-1:8, transferred to tissue culture plates previously coated with Matrigel for 2-4 hr in 37° C., and fed every 2-3 days with FMM. Cell cultures were maintained in a humidified incubator set at 37° C. and 5% CO₂.

Human iPSC Engineering with ZFN, CRISPR for Targeted Editing of Modalities of Interest:

Using ROSA26 targeted insertion as an example, for ZFN-mediated genome editing, 2 million iPSCs were transfected with a mixture of 2.5 μg ZFN-L (FTV893), 2.5 μg ZFN-R (FTV894) and 5 μg donor construct, for AAVS1 targeted insertion. For CRISPR-mediated genome editing, 2 million iPSCs were transfected with a mixture of 5 μg ROSA26-gRNA/Cas9 (FTV922) and 5 μg donor construct, for ROSA26 targeted insertion. Transfection was done using Neon transfection system (Life Technologies) using the following parameters: 1500V, 10 ms, 3 pulses. On day 2 or 3 after transfection, transfection efficiency was measured using flow cytometry to assess if the plasmids contain artificial promoter-driver GFP and/or RFP expression cassette. On day 4 after transfection, puromycin was added to the medium at a concentration of 0.1 μg/ml for the first 7 days and 0.2 μg/ml after 7 days to select the targeted cells. During the puromycin selection, the cells were passaged onto fresh matrigel-coated wells on day 10. On day 16 or later of puromycin selection, the surviving cells were analyzed by flow cytometry for GFP⁺ iPS cell percentage.

Bulk Sort and Clonal Sort of iPSCs:

iPSCs with or without genomic targeted editing were bulk sorted and clonal sorted for GFP⁺SSEA4⁺TRA181⁺ iPSCs after 20 days of puromycin selection. Single cell dissociated targeted iPSC pools were resuspended in staining buffer containing Hanks' Balanced Salt Solution (MediaTech), 4% fetal bovine serum (Invitrogen), 1× penicillin/streptomycin (Mediatech) and 10 mM Hepes (Mediatech). Conjugated primary antibodies, including SSEA4-PE, TRA181-Alexa, and Fluor-647 (BD Biosciences), were added to the cell solution. All antibodies were used at 7 μL in 100 μL staining buffer per million cells. The solution was washed once in staining buffer, spun down and resuspended in staining buffer containing 10 μM Thiazovivn for flow cytometry sorting. Flow cytometry sorting was performed on a FACS Aria II (BD Biosciences). For bulk sorting, GFP⁺SSEA4⁺TRA181⁺ cells were gated and sorted into FMM. For clonal sorting, the sorted cells were directly ejected into 96-well plates with each well coated with 5× Matrigel and prefilled with 200 μL FMM supplemented with 5 μg/mL fibronectin and 1× penicillin/streptomycin (Mediatech). Upon completion of the sort, 96-well plates were centrifuged for 1-2 min at 225 g prior to incubation. The plates were left undisturbed for seven days. On the seventh day, 150 μL of medium was removed from each well and replaced with 100 μL FMM. Wells were refed with an additional 100 μL FMM on day 10 post sort. Colony formation was detected as early as day 2 and most colonies were expanded between days 7-10 post sort. In the first passage, wells were washed with PBS and dissociated with 30 μL Accutase for approximately 10 min at 37° C. After cells are seen to be dissociating, the dissociated colony is transferred to another well of a 96-well plate previously coated with 5× Matrigel and then centrifuged for 2 min at 225 g prior to incubation. Subsequent passages were done routinely with Accutase treatment for 3-5 min and expansion of 1:4-1:8 upon 75-90% confluency into larger wells previously coated with 1× Matrigel in FMM. Each clonal cell line was analyzed for GFP fluorescence level and TRA1-81 expression level. Clonal lines with near 100% GFP⁺ and TRA1-81⁺ were selected for further PCR screening and analysis, and cryopreserved as a master cell bank.

### Hematopoietic Differentiation:

To initiate differentiation towards the hematopoietic lineage, hiPSCs were seeded as a monolayer on Day (D) 0 in the maintenance medium and allowed to adhere and expand for about 24 hours. The monolayers were maintained until around D5-D6 at which point they were dissociated into single cells and seeded as a low-density monolayer until differentiation at around D10. The D10 dissociated single cell population was sorted by FACS for further analysis and marker profiling for definitive HE characterization. Alternatively, or in addition thereto, as disclosed herein, one or more of anti-CD82, anti-CD34, and anti-CD43 antibodies were used for cell sorting the D10 cell population to obtain definitive HE cells comprising a phenotype of CD82⁺, CD34⁺CD82⁺ or CD34⁺CD43⁻CD82⁺, which were optionally cryopreserved in cryopreservation medium or continued with effector cell differentiation.

Differentiation of HE cells Towards T and NK Lineages:

Sorted D10 HE cells were further differentiated towards the iT and iNK lymphoid lineages. Specific to iT cells, upon sorting, the HE cells were transferred to low attachment tissue culture plates for serum-free differentiation. After approximately 10 days of culturing (post HE isolation) the cell culture was assessed for the generation of T cell progenitors by the co-expression of the cell surface markers CD34 and CD7. After further differentiation for approximately 15-20 days these CD34⁺CD7 T cell progenitors gave rise to distinct populations of mature iT cells as seen by the expression of CD4 and CD8.

Specific to iNK cells, upon sorting, the HE cells were cultured in serum-free differentiation media for approximately 10-15 days. The cell culture was assessed for the generation of NK cell progenitors. Following an additional 10-15 days of culturing and expansion, and optional modulation as further disclosed herein, the presence of activated/mature NK cells was identified using markers that include CD56, CD122, NKp30, CD94, CD16, NKG2D and KIR.

### Example 2—Definitive Hemogenic Endothelium Cell Surface Mark Screening

Cell markers serve as a monogram to help identify and classify cells. The majority of the markers are molecules or antigens within the plasma membrane of cells. Many surface markers are classified by their clusters of differentiation (CD) which are recognized by specific antibodies. Generally, specific combinations of markers are unique to different cell types.

During in vitro differentiation of pluripotent stem cells through mesoderm originated endothelium, the heterogeneous endothelial cells (ECs) acquire arterial, venous, or hemogenic fate, and form respective subtype endothelial cells which are phenotypically and functionally specialized. These cell subtypes are formed in close space and time, and are currently distinguished mainly through gene expression profiling. Hematopoietic cells arise from a unique population of endothelial cells known as hemogenic endothelium (HE) through an endothelial-hematopoietic transition (EHT). EHT represents a continuous process in which cells with endothelial characteristics gradually acquire hematopoietic morphology and phenotype, and the endothelial progenitors undergoing such hematopoietic transition are termed definitive hemogenic endothelium. Currently, definitive HE cells are identified as CD34⁺ cells that do not express CD43, CD73, CD93, and CXCR4. In comparison to this negative screen for a CD43⁻CD73⁻CD93⁻CXCR4⁻ cell subpopulation in the CD34⁺ cells, identifying one or more positive markers specific to definitive HE cell subtypes would greatly improve the efficiency and accuracy of isolating early cell populations with desired quality and purity for subsequent hematopoietic cell differentiation.

To effectively screen for novel positive markers of HE, a proprietary hiPSC platform of the applicant was used, which enables single cell passaging and high-throughput flow cytometry sorting, to allow for the derivation of clonal hiPSCs. To identify additional markers for the enrichment of definitive HE cells having the capacity to give rise to hematopoietic cells, hiPSCs were seeded as a monolayer and differentiated towards hematopoietic cells using the methods and compositions disclosed herein.

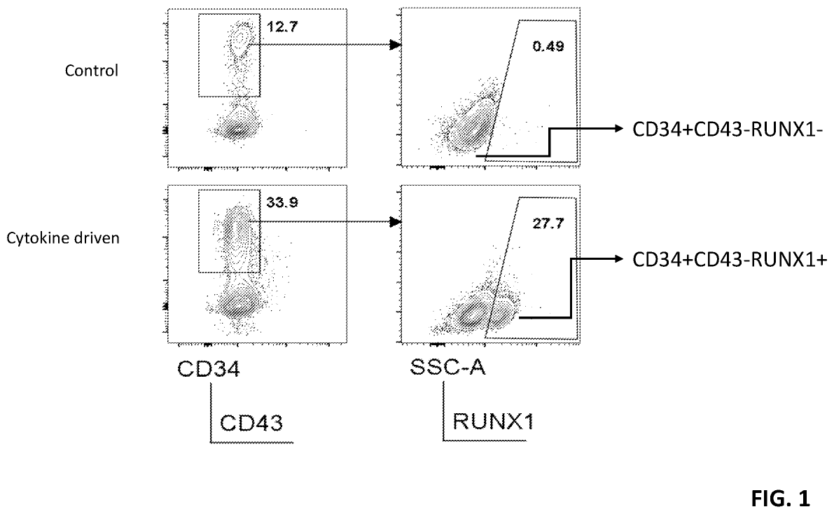

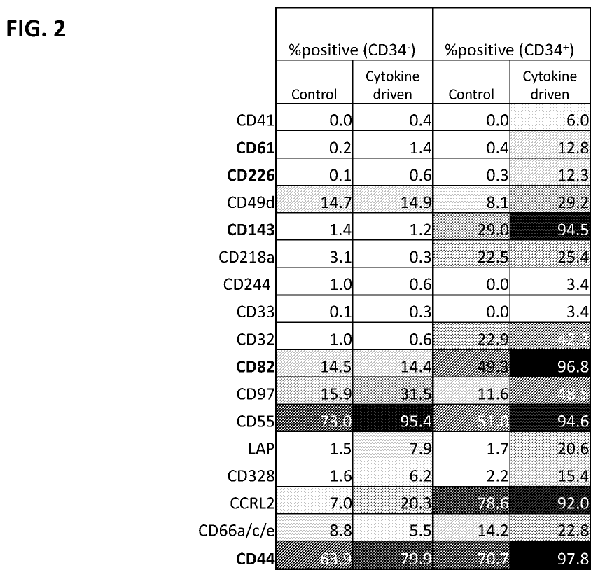

The flow cytometric analysis of D10 cells differentiated under control conditions was compared to cells differentiated under cytokine conditions that includes BMP4. It was discovered that the specification of RUNX1⁺ HE is cytokine driven as shown in FIG. 1. RUNX1 is a transcription factor expressed in the cell and believed to be mostly associated with HE identity. The cells were gated on single/live events. The CD34⁺RUNX1⁺ cells and the CD34⁺RUNX1⁺ cells were then stained with anti-human antibodies and analyzed by LEGENDScreen™. The screen subsequently identified a number of surface marker candidates that have a higher percentage of expression within the CD34⁺RUNX1⁺ subset compared to the control cells (FIG. 2). As shown in FIG. 2, CD82, CD61, CD44, CD143 and CD226, among other marker candidates shown, have higher expression in the CD34⁺ population than in the CD34⁻ population, and this higher expression in the CD34⁺ population also correlates to the cytokine driven RUNX1⁺ expression. Of the above marker candidates, CD143 and CD82 are most promising based on the differential expression and tight correlation with RUNX1 expression.

### Example 3—Definitive Hemogenic Endothelium Cell Surface Mark Verification

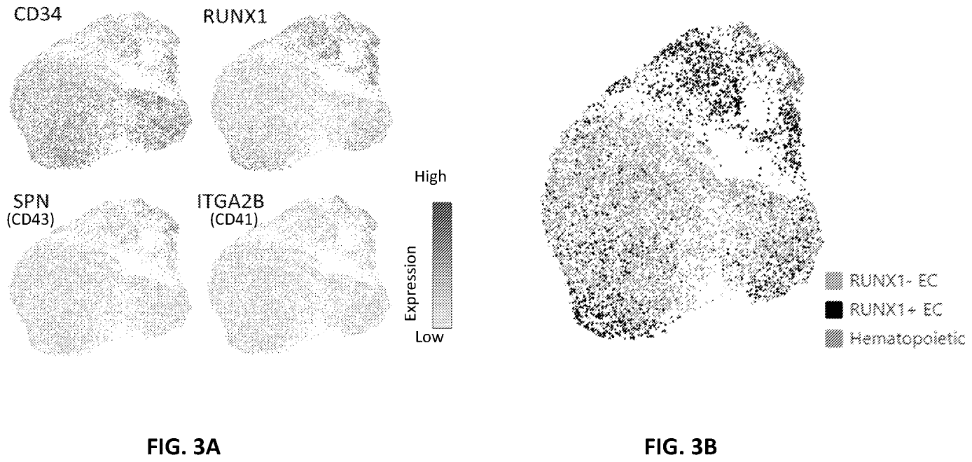

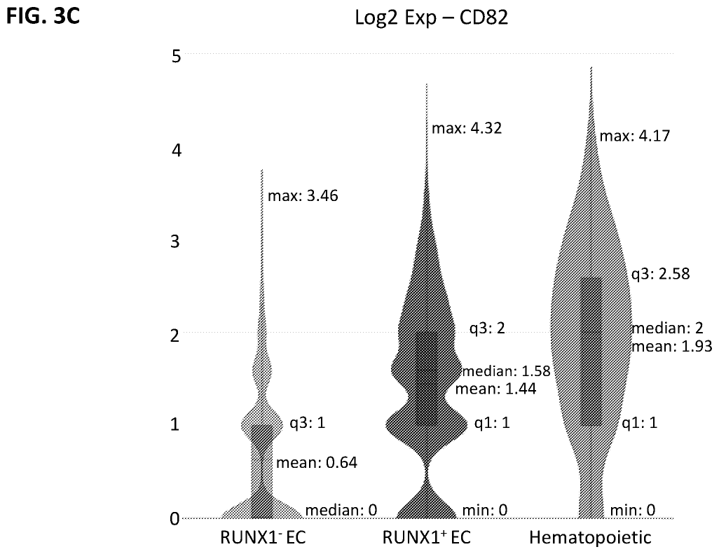

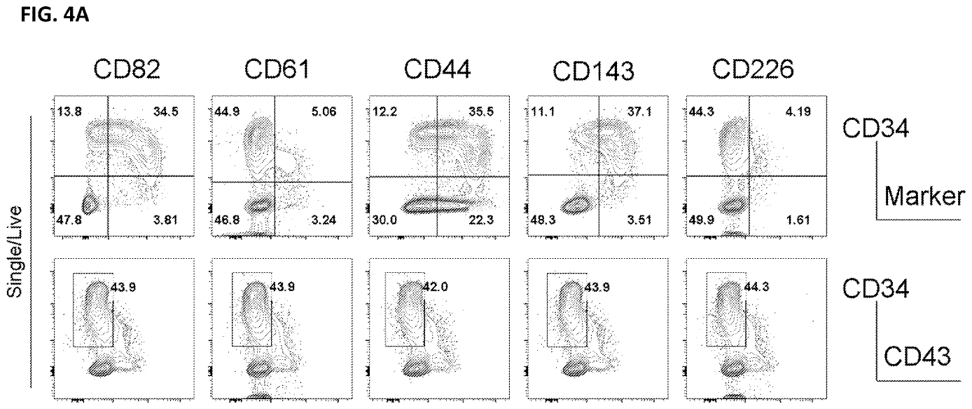

Cytokine driven D10 cells were purified using CD34 magnetic activated cell sorting. The scRNAseq analysis of curated feature gene expression is presented in FIG. 3A using UMAP (Uniform Manifold Approximation and Projection) visualization which was then used to identify cell clusters that express the respective feature genes (CD34, RUNX1, SPN or ITGA2B). As shown in FIG. 3A, each dot represents one cell, and the color density indicates the feature gene expression level.

The UMAP visualization of the curated feature genes of FIG. 3A was then used to define each cell cluster in FIG. 3B. The cell clusters in FIG. 3B include: (1) RUNX1⁻ ECs that express the endothelial marker CD34 but not RUNX1 or the hematopoietic markers SPN (CD43) or ITGA2B (CD41), which are non-hemogenic CD34⁺ cells; (2) RUNX1⁺ ECs that express CD34 and RUNX1 but not SPN or ITGA2B, which are the desirable HE cells; (3) any cells that express SPN or ITGA2B, which are (differentiated) hematopoietic cells.

Once the three cell clusters were identified in the D10 CD34⁺ cells, a violin plot was used to demonstrate the CD82 expression within each of the three subsets of the D10 cell population (FIG. 3C). As shown in FIG. 3C, with the width of the violin plot representing the number of cells at each CD82 expression level, and with the mean and medium CD82 expression labeled for each cell subset, the violin plot demonstrates that while hematopoietic cells are known to express both RUNX1 and CD82, CD82 gene expression is significantly higher in the RUNX1⁺ EC (HE) population compared to that in the RUNX1⁺ non-hemogenic population, thereby supporting the hypothesis that CD82 is a marker that can distinguish HE from non-HE.

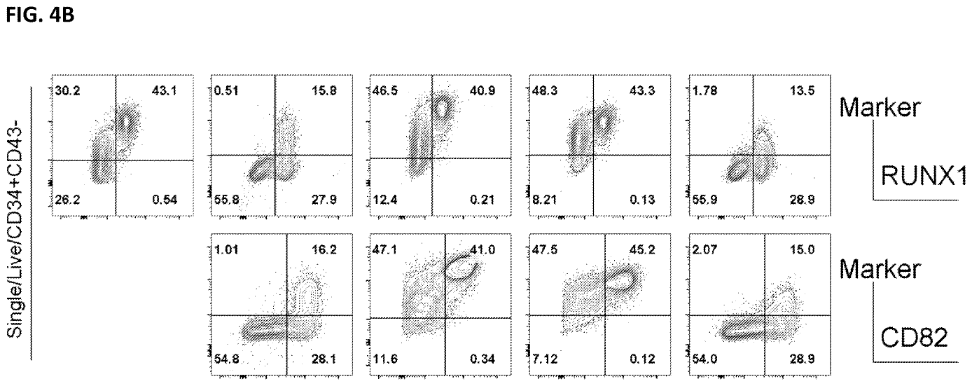

Flow cytometric analysis of cytokine driven D10 cells was subsequently performed to compare expression of other selected HE candidate markers with RUNX1 and CD82. Since RUNX1 is a transcription factor, and not cell surface expressed, it is not useful as a marker to enrich intact HE cells by flow sorting. As shown in FIG. 4A, the first row of flow plots depicts the candidate HE markers against CD34 to determine if the candidate marker is restricted to the endothelial population (CD34⁺); all tested markers except for CD44 are primarily restricted to the endothelial population. The second row (FIG. 4A) shows CD34 against CD43 to identify the CD34⁺CD43⁻ population. The third and fourth rows (FIG. 4B) depict the candidate HE markers gated through the CD34⁺CD43″ population against RUNX1 (third row) and CD82 (fourth row). As shown, all candidate markers are expressed within the RUNX1⁺ population. It was observed that CD61 and CD226 are not expressed by the entire RUNX1⁺ population suggesting that they mark only a subset of the HE at D10 of differentiation. Since CD82 and CD143 are both restricted to the endothelial population and expressed within the RUNX1⁺ population, the data support each of them as viable HE candidate surface markers.

A further observation was made that the addition of a p38 MAPK (mitogen-activated protein kinase) inhibitor to the composition for obtaining definitive HE cells from iPSC is beneficial to maintain CD82 expression, leading to the maintenance of CD82⁺RUNX1⁺ subpopulation of CD34⁺ HE cells. Therefore, it is contemplated that in addition to BMP4 cytokine, the composition may optionally further comprise a p38 MAPK inhibitor, such as DBM1285 (an exemplary small molecule inhibitor that was tested), to increase the efficiency of obtaining definitive HE cells from iPSC differentiation, wherein the definitive HE cells have a phenotype comprising CD82⁺, and optionally one or more of CD34⁺, CD43⁻, CD93⁻, CXCR4⁻, and CD73⁻.

### Example 4—Definitive Hemogenic Endothelium Cell Surface Mark Application

Current methods for identifying definitive HE relies on the exclusion of endothelial populations (CD34⁺CD43⁻) that express more mature endothelial markers including CD73, CD93 and CXCR4, such that the selected HE generally has a profile of CD34⁺CD43⁻CD73⁻CD93⁻CXCR4⁻. However, this phenotype is also shared by endothelial progenitor cells that precede HE in cell development. Since cells typically differentiate slowly, the population having a higher percentage of CD34⁺CD43⁻CD73⁻CD93⁻CXCR4-would weigh more towards immature endothelial progenitor cell development rather than definitive HE, thereby making the negative marker screening an unreliable method for the detection of definitive HE cells which have the potential to develop into T lineage cells in addition to NK, NKT and B cell lineages.

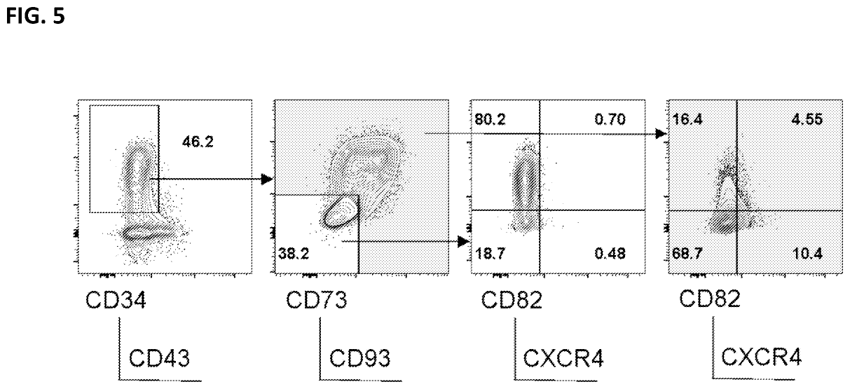

For the CD82 and CD143 markers from the above screening, CD82 was used as a demonstration to show their validity as definitive HE markers. The flow cytometric analysis of cytokine driven D10 cells was performed to demonstrate enrichment of CD82⁺ cells within a CD73⁻CD93⁻CXCR4⁻ endothelial population, with cells pre-gated on single/live events. As shown in FIG. 5, CD82 expression is restricted within the CD34⁺CD43⁻CD73⁻CD93⁻CXCR4⁻ population of cells, and the data therefore supports that CD82 is a reliable marker replacing the triple-negative marker for identifying definitive HE.

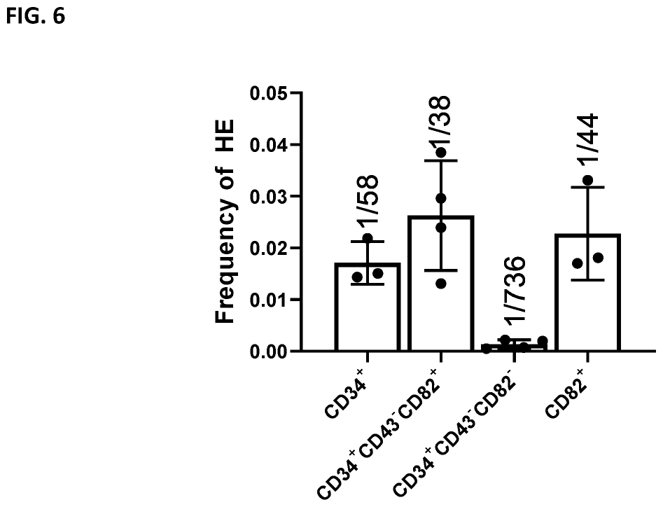

In a separate experiment, the frequency of definitive HE within cytokine driven D10 populations was calculated using extreme limiting dilution analysis (ELDA) software. Cytokine driven D10 populations were fluorescence-activated cell sorted (FACs) based on markers indicated along the x-axis (mean±SD) and the average definitive HE frequency is indicated above each column in FIG. 6. Significantly, 1 in 38 cells of the CD34⁺CD43⁻CD82⁺ population and 1 in 44 of the CD82⁺ population were definitive HE, which further supports that CD82 can be used as a reliable positive marker for definitive HE identification. Meanwhile, the data also show that the D10 cell sorting for definitive HE may use a single marker CD82⁺, or use CD43⁺CD82⁺ (eliminating differentiated hematopoietic cells by CD43⁻), or use CD34⁺CD43⁻CD82⁺ for obtaining a satisfactory definitive HE frequency that is each improved upon the CD34⁺ cell population.

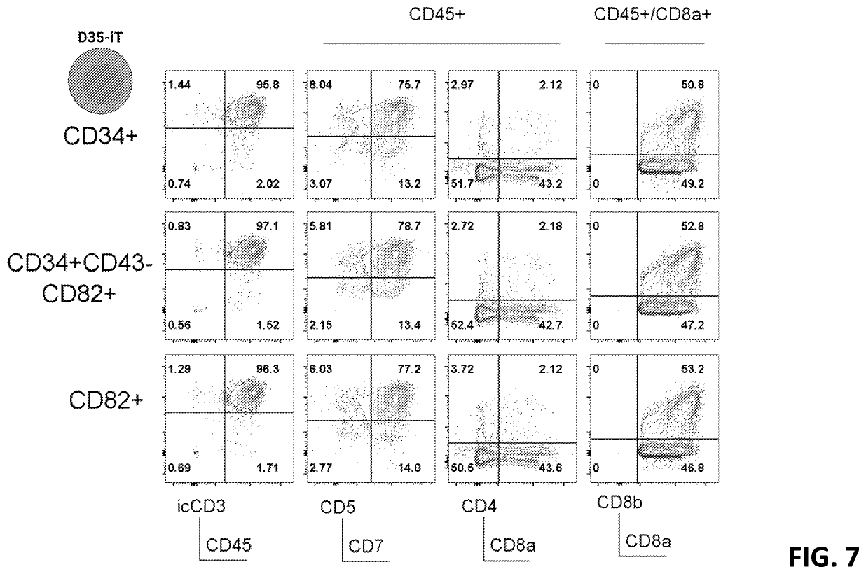

The determination of the definitive hematopoietic potential represented by cell populations obtained using various marker combinations was through iT differentiation. The D10 CD34⁺CD43⁻CD82⁺ and CD34⁺CD43⁻CD82; cell fractions isolated by FACS were plated in cultures (iTC-A2 and iTC-B2) as disclosed herein. At about D35 of iT differentiation, the cultures were assessed for the presence of iT cells by the expression of the pan-hematopoietic marker CD45, along with the lymphoid markers CD5 and CD7. Since the iT cells in this example have CD19-CAR knocked into the TRAC locus, preventing assembly of the T cell receptor on the cell surface, the detectable intracellular T cell co-receptor CD3 (icCD3) was used as a marker for differentiated T cells. As shown in FIG. 7, populations were FAC sorted by markers indicated above the flow plots, and the iT cells differentiated from each indicated D10 populations gave rise primarily to CD8⁺ cytotoxic T cells, but not CD4⁺ helper T cells. Further, without being limited by theory, expression of CD8ab (CD8a⁺CD8b⁺) suggests that the cells are more adaptive, as compared to CD8aa (CD8a⁺CD8b⁻) which is primarily expressed by innate-like T cells. The successful differentiation of T cells from the CD82⁺ fraction of D10 sorted CD34⁺CD43⁻ cells is indicative of the definitive nature of the HE cells identified by CD82.

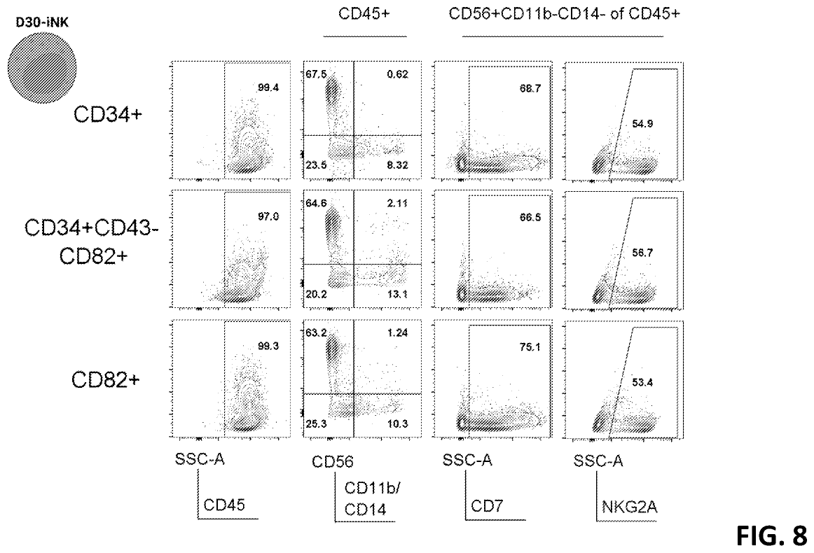

For iNK differentiation, the D30 cultures were assessed for the presence of iNK cells by the expression of the pan-hematopoietic marker CD45 and the NK marker CD56. Populations were FAC sorted by markers indicated above the flow plots of FIG. 8. While relatively small populations of myeloid cells (CD11b/CD14⁺) were detected, the robust expression of the lymphoid marker CD7 and expression of the NK cell receptor NKG2A shown in FIG. 8 indicates successful NK cell differentiation.

Collectively, the data demonstrate that CD82 is a reliable cell surface marker for definitive HE detection during iPSC differentiation. Further, the CD82⁺ or CD34⁺CD82⁺ cells represent the definitive HE population in an early stage of iPSC directed differentiation towards hematopoietic cells; and the expression of CD82 can be used as a surrogate positive marker to replace the negative markers including CD73, CD93, CXCR4 or any combinations thereof in identifying HE, or more specifically, definitive HE, subpopulations.

### Example 5—OP9 Stromal-Free Differentiation of HE Towards Mature and Functional Lymphoid Cells

In an effort to differentiate iPSCs without murine- or human-derived stromal cells in chemically defined and serum-free media, the sorted definitive HE cells (iCD34; CD82⁺; CD34⁺CD82⁺; or CD34⁺CD43⁻CD82⁺) were seeded in a medium without passaging for about 18-20 days, or up to 27-30 days, before the differentiated CD56 expressing NK cells were ready for collection, storage, or further maturation and expansion. Notably, this differentiation process as described herein is entirely free of feeder cells, specifically, the DLL4-expressing OP9 stromal cells (OP9-DLL4) or their irradiated counterparts (irOP9-DLL4), rendering the differentiated cell product free of exogenous undefined cell components, which is desirable for regulatory compliance in producing a cell therapy.

The composition replacing OP9-DLL4 for hematopoietic cell, including NK cell, differentiation comprises human DLL4 Fc chimera recombinant protein (Fc-rhDLL4) and RetroNectin® (Takara Bio USA, Inc., San Jose, CA). RetroNectin in this Retro/DLL4 extracellular matrix is a recombinant human fibronectin having three functional domains: the human fibronectin cell-binding domain (C-domain), heparin-binding domain (H-domain), and CS-1 sequence domain. The medium comprising the Retro/DLL4 extracellular matrix further comprises IL7, Flt3L and SCF, and optionally one or more of a ROCK inhibitor, IL3, TPO, and VEGF. For example, the ROCK inhibitor may be present in the medium for the first 2-4 days, TPO and VEGF may be present in the medium for the first 4-8 days, and IL3 may be present in the medium for the first 7-11 days, after iCD34 cells are seeded.

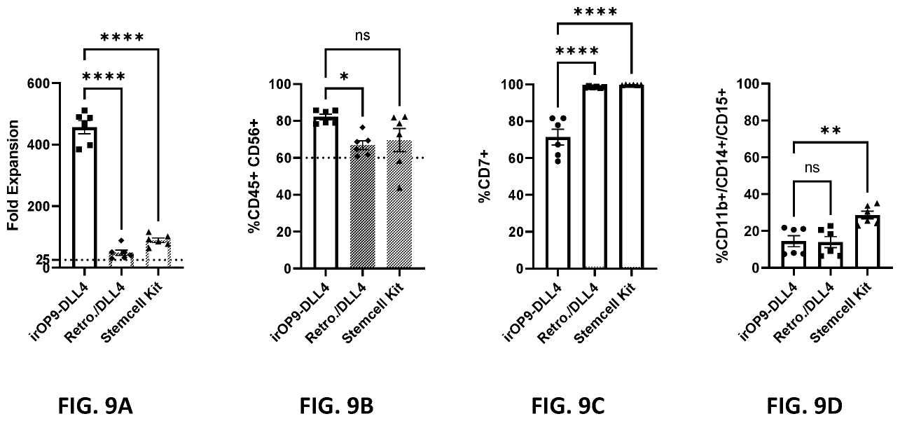

The stromal-free differentiation of definitive HE cells was compared to those using irOP9-DLL4 or commercially available NK differentiation Stemcell Kit (Stemcell Technologies) for iNK fold expansion and iNK progenitor specification/profile depicted by frequency of CD45⁺CD56⁺, CD7⁺ and CD11b⁺/CD14⁺/CD15⁺ cells. As shown in FIGS. 9A-9D, all three differentiation strategies met minimum fold expansion (i.e., ˜25-fold), and produced differentiated cells having similar iNK progenitor specifications (for example, CD45⁺CD56⁺ and CD7⁺ frequencies). While the Retro/DLL4 differentiation may not have cell expansion as robust as seen under the irOP9-DLL4 differentiation, it is superior to the Stemcell Kit for producing fewer myeloid cells (for example, CD11b⁺/CD14⁺/CD15⁺ frequency).

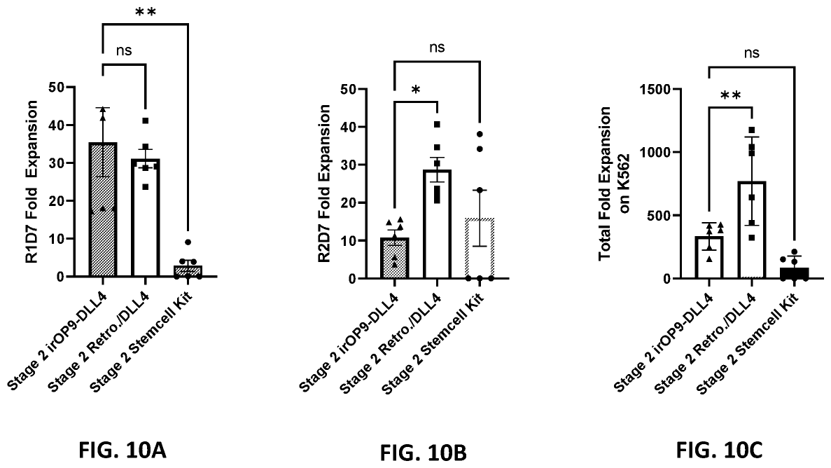

The collected iNK progenitor cells were combined with irradiated K562 cells engineered to express 41BBL and IL21 (irK562-41BBL-IL21) in a tissue culture vessel to mature and expand over two rounds of a 7-day expansion period. After the first 7-day maturation and expansion period (R1D7), the cells were assayed for fold expansion and restimulated with additional irK562-41BBL-IL21 to continue maturation and expansion. After the second 7-day maturation and expansion period (R2D7), the cells were assayed for fold expansion and cryopreserved for in vitro cytotoxicity assays. As shown in FIGS. 10A-10C, while the iNK progenitors differentiated using the Stemcell Kit failed to expand after R1D7, those differentiated on irOP9-DLL4 and Retro/DLL4 displayed similar levels of expansion far exceeding the Stemcell Kit differentiation. Moreover, after R2D7, the Retro/DLL4 group surprisingly displayed superior expansion over both the irOP9-DLL4 and Stemcell Kit groups, resulting in a much more desirable total fold expansion over both rounds of maturation and expansion (FIG. 10C). Further, the mature expanded CD45⁺CD56⁺ iNK cells from the Retro/DLL4 group and those obtained using conventional irOP9-DLL4 differentiation displayed a similar profile depicted by various inhibitory, activating and co-activating NK receptors, thereby demonstrating that the stromal-free Retro/DLL4 system is an effective and efficient solution for differentiating HE cells towards NK lineage cells.

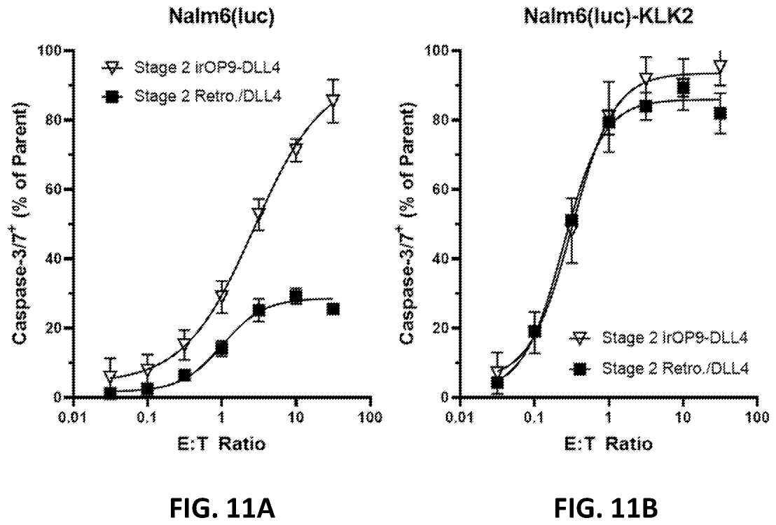

To demonstrate the functionality of the mature iNK cells differentiated from definitive HE cells using the stromal-free method, the cryopreserved iNK cells from the irOP9-DLL4 and Retro/DLL4 groups were each thawed and co-cultured with Nalm6 target cells or Nalm6 cells engineered to express an exemplary surface antigen (Nalm6-KLK2, for example). After four hours of co-culture, a caspase 3/7 activity assay was used to measure cellular apoptosis. As shown in FIGS. 11A and 11B, even though the iNK cells of the Retro/DLL4 group showed lower antigen-independent spontaneous killing of Nalm6 target cells over a range of effector:target ratios than those of the irOP9-DLL4 group, iNK cells of both groups have equally effective antigen-specific killing directed by a CAR. The lower antigen-independent spontaneous killing of iNK cells differentiated from the stromal-free Retro/DLL4 method could be advantageous for therapeutic effector cells as a desirable safety attribute by reducing unspecified innate NK killing.

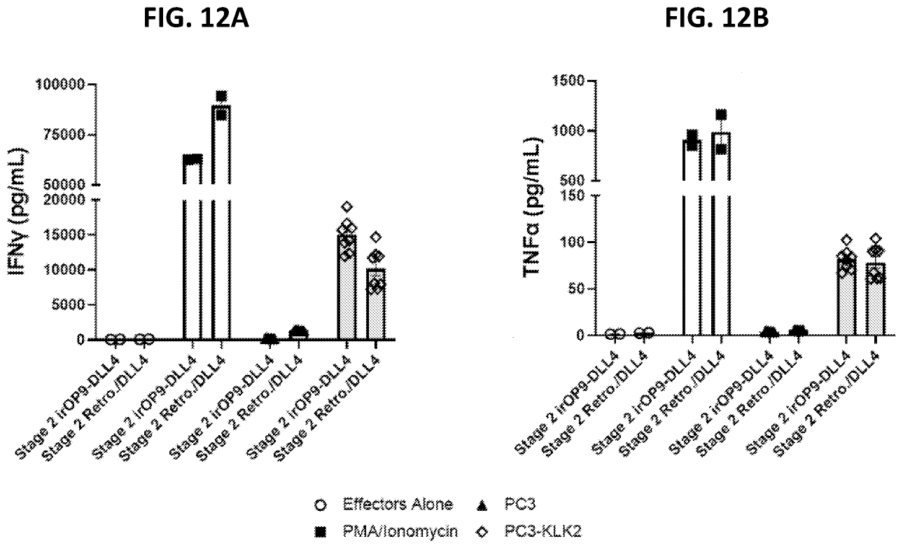

In a separate experiment, the thawed iNK cells from the irOP9-DLL4 and Retro/DLL4 groups were co-cultured with PC3 cells or PC3 cells engineered to surface express an antigen (PC3-KLK2, for example). After 16 hours, cytokine release in each group was measured in supernatant by ELISA. As shown in FIGS. 12A and 12B, the iNK cells from both groups displayed similar levels of maximal release of IFNγ and TNFα under the PMA/Ionomycin stimulation, as well as similar levels of antigen-dependent cytokine release of IFNγ and TNFα, confirming that the stromal-free Retro/DLL4 differentiation system supports the functionality of differentiated effector cells.

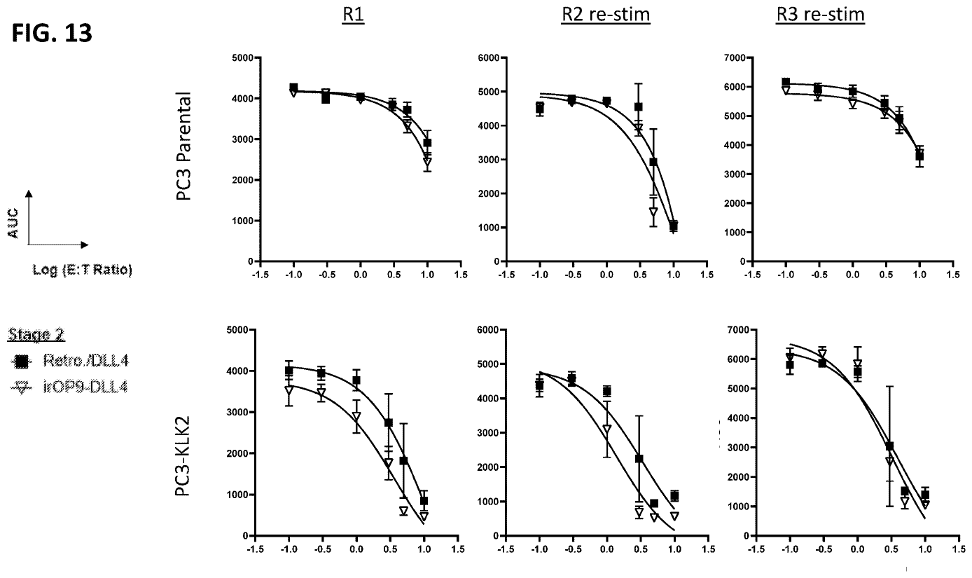

To demonstrate persistency of the differentiated effector cells, the mature expanded iNK cells from the irOP9-DLL4 and Retro/DLL4 groups were further subjected to an IncuCyte® serial re-stimulation assay. In this assay, the iNK cells differentiated from each group were combined with PC3 parental cells or cell surface antigen expressing PC3 cells (PC3-KLK2, for example) at multiple E:T (Effector:Target) ratios. The number of viable target cells was monitored by hourly fluorescence imaging over 48 hours. Live cell numbers were quantified and normalized to the number of live cells remaining in the target cell-only control group. After 48 hrs, iNK cells from R1 were re-stimulated with fresh PC3 parental or PC3-KLK2 target cells to generate normalized live cell counts (R2 re-stim). The iNK cells from R2 were again re-stimulated after 48 hours to generate normalized live cell counts (R3 re-stim). As shown in FIG. 13, the iNK cells from the Retro/DLL4 group maintained similar capability in antigen-dependent serial killing of target cells as those from the irOP9-DLL4 group, further verifying that the stromal-free Retro/DLL4 differentiation system supports long-term effector cell functionality.

### Example 6—Modified Expansion Composition of Mature Lymphoid Cells

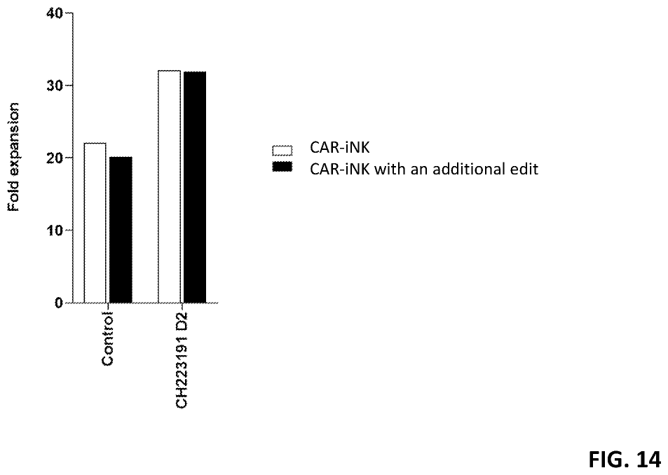

To modulate effector cell activation during the cell expansion stage of iPSC differentiation, an AhR (aryl hydrocarbon receptor) inhibitor was discovered to improve effector cell function, including anti-tumor efficacy. iPSCs were differentiated into definitive HE cells as previously described, and were subsequently differentiated into pre-expansion NK cells, at which point they were expanded by co-culture with IL21 and 41BBL expressed by irK562 feeder cells for about 7 days. An exemplary AhR inhibitor, CH-223191 (among others such as UM729, UM171, SR1), was added to the expansion culture at day 2 with a final concentration of about 3 μM to potentiate expansion signals. FIG. 14 shows that in the presence of CH-223191 a greater fold expansion and yield of differentiated NK cells are achieved in comparison to iNK cells expanded without the small molecule AhR inhibitor.

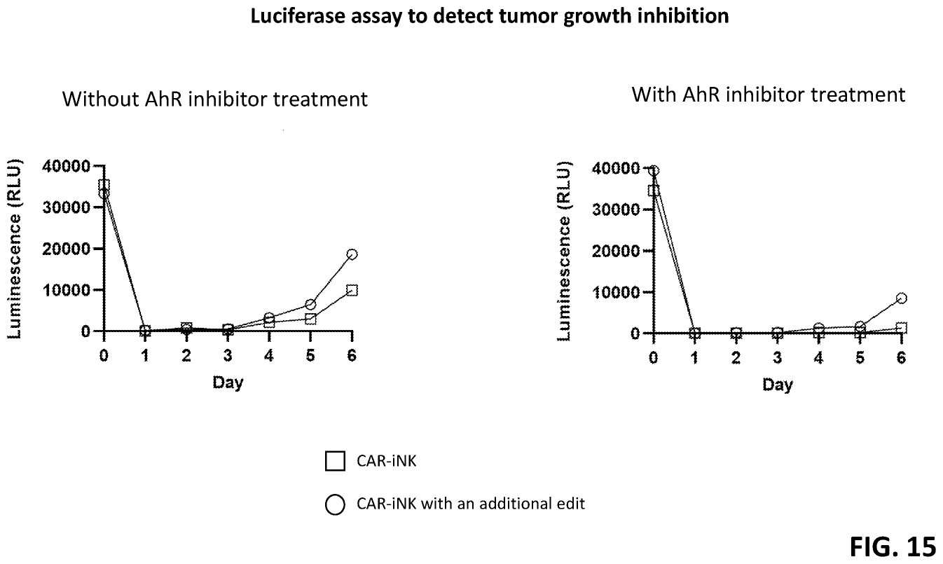

Cells that have been treated with CH-223191 were cryopreserved in liquid nitrogen long term. To assess recovery of the cells from cryopreservation, the cells were thawed and cultured in B0 medium for a period of 7 days and cell counts taken during the course of this culture. Cells treated with the AhR inhibitor showed greater post thaw recovery and persistence over time. Further, cells that have been treated with CH-223191 and cryopreserved in liquid nitrogen were further assessed for function in vitro and in vivo. Cells that have been treated with CH-223191 and cryopreserved in liquid nitrogen showed enhanced anti-tumor efficacy at endpoint compared to untreated or control cells, demonstrating that AhR inhibitor treatment resulted in better anti-tumor efficacy over time (FIG. 15).

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A cell population comprising cells having a phenotype of:
(i) CD82⁺;
(ii) CD34⁺CD82⁺; and/or
(iii) CD34⁺CD43⁻CD82⁺,
wherein the cells comprise definitive hemogenic endothelium (HE) cells, and wherein the cells are derived from iPSC differentiation in vitro.

2. The cell population of claim 1, wherein the definitive HE cells
(i) are enriched; and/or
(ii) are capable of differentiating into hematopoietic lineage cells comprising T cell progenitors and T cells, in addition to NK progenitors, NK cells, NKT cells, or B cells.

3. The cell population of claim 1, wherein the iPSC is a clonal iPSC, a single cell dissociated iPSC, an iPSC cell line cell, or an iPSC master cell bank (MCB) cell.

4. The cell population of claim 3, wherein the iPSC is a naïve iPSC.

5. The cell population of claim 3, wherein the iPSC further comprises one or more genetic imprints introduced to the iPSC by genomic editing during or after reprogramming a non-pluripotent cell to the iPSC, wherein the genetic imprint comprises:
(i) one or more genetically modified modalities introduced through genomic insertion, deletion or substitution in the genome of the iPSC; or
(ii) one or more retainable therapeutic attributes of a source specific immune cell that is donor-, disease-, or treatment response-specific, and wherein the iPSC is reprogrammed from the source specific immune cell;
and wherein the cells comprise the same one or more genetic imprints.

6. The cell population of claim 1, wherein the iPSC differentiation to obtain the cell population comprises:
(i) differentiating iPSCs to obtain hemogenic endothelium (HE) cells, and
(ii) sorting HE cells for cells that are CD82⁺ to obtain definitive HE cells expressing cell markers comprising CD82⁺,
wherein the definitive HE cells are capable of differentiating into hematopoietic lineage cells.

7. The cell population of claim 6, wherein differentiating iPSCs to obtain HE cells further comprises:
(a) differentiating iPSCs to obtain mesoderm progenitors; and
(b) differentiating the mesoderm progenitors to obtain HE cells.

8. The cell population of claim 6, wherein the cell markers further comprise CD34⁺, CD43⁻, RUNX1⁺, or any combinations thereof, and wherein the obtained definitive HE cells comprise a phenotype of CD34⁺CD82⁺, CD34⁺CD43⁻CD82⁺, CD34⁺CD82⁺RUNX1⁺, or CD34⁺CD43⁻CD82⁺RUNX1⁺.

9. The cell population of claim 6, wherein the iPSC differentiation comprises contacting the iPSCs with:
(i) a cytokine that leads to a higher percentage of RUNX1 expressing HE cells as compared to without the cytokine; and/or
(ii) a small molecule p38 MAPK (mitogen-activated protein kinase) inhibitor that leads to improved maintenance of CD82 expression in HE cells as compared to without the inhibitor.

10. The cell population of claim 9, wherein the cytokine comprises BMP4, and/or wherein the small molecule p38 MAPK inhibitor comprises DBM1285.

11. The cell population of claim 1, wherein (i) at least 0.5%, at least 1%, or at least 2% of the cells that are CD82⁺ are definitive HE cells; and/or (ii) the cell population is a substantially pure population of the cells having the phenotype.

12. A composition comprising the cell population of any one of the claims 1-11.

13. The composition of claim 12, further comprising a cryopreservation medium.

14. A method of generating iPSC-derived definitive HE, wherein the method comprises differentiating iPSC to obtain iPSC-derived hemogenic endothelium (HE) cells and sorting the HE cells for cells that are CD82⁺, thereby obtaining definitive HE cells expressing cell markers comprising CD82⁺,
wherein the definitive HE cells are capable of differentiating into hematopoietic lineage cells comprising T cell progenitors and T cells, in addition to, NK progenitors, NK cells, NKT cells, or B cells.

15. The method of claim 14, wherein the sorting further comprises sorting for cells that are CD34⁺, CD43⁻, RUNX1⁺, or any combinations thereof; and wherein the obtained definitive HE cells comprise a phenotype of CD34⁺CD82⁺, CD34⁺CD43⁻CD82⁺, CD34⁺CD82⁺RUNX1⁺, or CD34⁺CD43⁻CD82⁺RUNX1⁺.

16. The method of claim 14, wherein the method further comprises:
(i) contacting the iPSCs with a medium comprising a BMP activator and bFGF, thereby differentiating the iPSCs to obtain mesoderm progenitors; and
(ii) contacting the mesoderm progenitors with a medium comprising a BMP activator, bFGF, VEGF, a Wnt pathway activator and optionally a p38 MAPK inhibitor, thereby differentiating the mesoderm progenitors to obtain HE cells.

17. The method of claim 16, wherein contacting with the p38 MAPK inhibitor increases maintenance of CD82 expression in HE cells as compared to without the p38 MAPK inhibitor; wherein the BMP activator comprises BMP4; and/or wherein the Wnt pathway activator comprises a GSK3 inhibitor.

18. The method of claim 17, wherein the p38 MAPK inhibitor comprises DBM1285; and/or wherein the GSK3 inhibitor comprises CHIR99021.

19. The method of claim 16, wherein the iPSCs comprise naïve iPSCs, and/or are derived from iPSCs comprising one or more genetic imprints.

20. The method of claim 19, wherein the one or more genetic imprints comprised in the iPSCs are retained in the iPSC-derived definitive HE cells.

21. The method of claim 14, further comprising cryopreserving the definitive HE cells.

22. A composition for generating iPSC-derived definitive HE (hemogenic endothelium) cells comprising: a BMP activator, bFGF, VEGF, a Wnt pathway activator, and optionally a p38 MAPK inhibitor.

23. The composition of claim 22, wherein:
(i) the composition is free of TGFβ receptor/ALK inhibitors;
(ii) the iPSC-derived definitive HE comprises increased RUNX1-expressing cells as compared to differentiation without the BMP activator; and/or
(iii) the iPSC-derived definitive HE comprises increased CD82-expressing cells as compared to differentiation without the p38 MAPK inhibitor.

24. The composition of claim 22, wherein the BMP activator comprises BMP4; and/or wherein the p38 MAPK inhibitor comprises at least one of DBM1285, VX-745, VX-702, RO-4402257, SCIO-469, BIRB-796, SD-0006, PH-797804, AMG-548, LY2228820, SB-681323, GW-856553, RV568, CAS 219138-24-6, SB203580, and SB242235.

25. The composition of claim 22, wherein the p38 MAPK inhibitor comprises DBM1285.

26. The composition of claim 22, further comprising iPSCs, mesodermal cells, or the definitive HE cells.

27. A method of generating iPSC-derived definitive HE comprising:
(i) differentiating iPSCs to obtain mesoderm progenitor cells;
(ii) differentiating the mesoderm progenitor cells to obtain HE cells; and
(iii) sorting the HE cells for cells that are CD82⁺ to obtain definitive hemogenic endothelium (HE) cells expressing cell markers comprising CD82⁺,
wherein the definitive HE cells are capable of differentiating into hematopoietic lineage cells comprising T cell progenitors and T cells, in addition to NK progenitors, NK cells, NKT cells, or B cells.

28. The method of claim 27, wherein the sorting further comprises sorting for cells that are CD34⁺, CD43⁻, RUNX1⁺, or any combinations thereof, and wherein the obtained definitive HE cells comprise a phenotype of CD34⁺CD82⁺, CD34⁺CD43⁻CD82⁺, CD34⁺CD82⁺RUNX1⁺, or CD34⁺CD43⁻CD82⁺RUNX1⁺.

29. The method of claim 27, wherein the step (ii) differentiating the mesoderm progenitor to HE cells comprises contacting the mesoderm progenitor with:
(i) a cytokine that leads to a higher percentage of RUNX1 expressing HE cells as compared to without the cytokine; and/or
(ii) a small molecule p38 MAPK (mitogen-activated protein kinase) inhibitor that leads to improved maintenance of CD82 expression in HE cells as compared to without the inhibitor.

30. The method of claim 29, wherein the cytokine comprises BMP4; and/or wherein the small molecule p38 MAPK inhibitor comprises DBM1285.

31. The method of claim 27, further comprising cryopreserving the obtained definitive HE cells.

32. A method of generating iPSC-derived hematopoietic lineage cells by differentiating the definitive HE cells of any one of the claims 1-11, wherein the method comprises contacting the definitive HE cells with a medium composition comprising SCF, Flt3L, and IL7; and optionally one or more of a ROCK inhibitor, TPO, and IL3, thereby obtaining the iPSC-derived hematopoietic lineage cells comprising T cell progenitors and T cells, in addition to NK cell progenitors, NK cells, NKT cells, or B cells.

33. The method of claim 32, wherein the iPSC-derived hematopoietic lineage cells comprise NK cell progenitors, and/or NK cells, and wherein (1) the medium composition further comprises IL15; and/or (2) the definitive HE cells comprise a genetic insertion of a polynucleotide encoding a cytokine signaling complex comprising a partial or full peptide of cell surface expressed exogenous IL 15 and/or a receptor thereof.

34. The method of claim 33, wherein the medium composition is free of OP9 stromal cells.

35. The method of claim 34, wherein the differentiating occurs in the presence of an extracellular matrix comprising a recombinant human fibronectin or fragment thereof and Fc-rhDLL4.

36. A method of manufacturing iPSC-derived hematopoietic lineage cells comprising:
differentiating iPSCs to obtain definitive hemogenic endothelium (HE) cells, wherein the definitive HE cells express cell markers comprising CD82⁺; and
differentiating the definitive HE cells to obtain iPSC-derived hematopoietic lineage cells;
wherein the iPSC-derived hematopoietic lineage cells comprise T cell progenitors and T cells, in addition to NK cell progenitors, NK cells, NKT cells, or B cells.

37. The method of claim 36, wherein the iPSCs comprise one or more genetic imprints introduced to the iPSCs by genomic editing during or after reprogramming non-pluripotent cells to the iPSCs, wherein the one or more genetic imprints comprise:
(i) one or more genetically modified modalities introduced through genomic insertion, deletion or substitution in the genome of the iPSCs; or
(ii) one or more retainable therapeutic attributes of source specific immune cells that are donor-, disease-, or treatment response-specific, wherein the iPSCs are reprogrammed from the source specific immune cells, and
wherein the one or more genetic imprints is retained in the iPSC-derived hematopoietic lineage cells.

38. The method of claim 36, wherein differentiating the iPSCs to obtain definitive hemogenic endothelium (HE) cells comprises:
(i) differentiating genetically engineered iPSCs to obtain mesoderm progenitors;
(ii) differentiating the mesoderm progenitors to obtain HE cells; and
(iii) sorting HE cells for cells that are CD82⁺ to obtain the definitive HE cells expressing cell markers comprising CD82⁺.

39. The method of claim 38, wherein the sorting further comprises sorting for cells that are CD34⁺, CD43⁻, RUNX1⁺, or any combinations thereof, and wherein the obtained definitive HE cells comprise a phenotype of CD34⁺CD82⁺, CD34⁺CD43⁻CD82⁺, CD34⁺CD82⁺RUNX1⁺, or CD34⁺CD43⁻CD82⁺RUNX1⁺.

40. The method of claim 38, wherein differentiating the mesoderm progenitor to HE cells comprises contacting the mesoderm progenitor with:
(i) a cytokine that leads to a higher percentage of RUNX1 expressing HE cells as compared to without the cytokine; and/or
(ii) a small molecule p38 MAPK (mitogen-activated protein kinase) inhibitor that leads to improved maintenance of CD82 expression in HE cells as compared to without the inhibitor.

41. The method of claim 40, wherein the cytokine comprises BMP4; and/or wherein the small molecule p38 MAPK inhibitor comprises DBM1285.

42. The method of claim 36, further comprising cryopreserving the definitive HE cells, wherein the cryopreserved definitive HE cells are thawed prior to differentiation thereof.

43. The method of claim 36, wherein differentiating the definitive HE cells is free of OP9 stromal cells.

44. The method of claim 43, wherein differentiating the definitive HE cells occurs in the presence of an extracellular matrix comprising a recombinant human fibronectin or fragment thereof and Fc-rhDLL4.

45. A method of generating an NK cell in a feeder-free environment comprising:
(a) differentiating an iPSC or definitive HE cell derived therefrom to NK lineage cells in a culture medium comprising one or more growth factors and cytokines comprising SCF, Flt3L, and IL7; wherein the culture medium is free of OP9 stromal cells; and further wherein:
(i) the culture medium comprises IL15, and/or
(ii) the definitive HE cells comprise a genetic insertion of a polynucleotide encoding a cytokine signaling complex comprising a partial or full peptide of cell surface expressed exogenous IL15 and/or a receptor thereof; and
(b) expanding and activating the NK lineage cells to obtain NK cells having cytotoxicity against a target.

46. The method of claim 45, wherein the culture medium further comprises one or more of a ROCK inhibitor, TPO, and IL3.

47. The method of claim 45, wherein the definitive HE cell comprises a phenotype comprising:
(i) CD82⁺;
(ii) CD34⁺CD82⁺;
(iii) CD34⁺CD43⁻CD82⁺; and/or
(iv) CD34⁺, and at least one of CD43⁻, CD93⁻, CXCR4⁻, CD73⁻, and RUNX1⁺.

48. The method of claim 45, wherein differentiating the iPSC further comprises:
(i) differentiating the iPSC to obtain mesoderm progenitors;
(ii) differentiating the mesoderm progenitors to obtain hemogenic endothelium (HE) cells; and
(iii) sorting HE cells for cells that are CD82⁺ to obtain the definitive HE cells, wherein the definitive HE cell expresses cell markers comprising CD82⁺.

49. The method of claim 48, wherein the sorting further comprising sorting for cells that are CD34⁺, CD43⁻, RUNX1⁺, or any combinations thereof, and wherein the obtained definitive HE cells comprise a phenotype of CD34⁺CD82⁺, CD34⁺CD43⁻CD82⁺, CD34⁺CD82⁺RUNX1⁺, or CD34⁺CD43⁻CD82⁺RUNX1⁺.

50. The method of claim 48, wherein differentiating the mesoderm progenitor to HE cells comprises contacting the mesoderm progenitor with:
(i) a cytokine that leads to a higher percentage of RUNX1 expressing HE cells as compared to without the cytokine; and/or
(ii) a small molecule p38 MAPK (mitogen-activated protein kinase) inhibitor that leads to improved maintenance of CD82 expression in HE cells as compared to without the inhibitor.

51. The method of claim 50, wherein the cytokine comprises BMP4; and/or wherein the small molecule p38 MAPK inhibitor comprises DBM1285.

52. The method of claim 45, wherein step (a) differentiating further comprises contacting the definitive HE cells with an extracellular matrix comprising a recombinant human fibronectin or fragment thereof and Fc-rhDLL4; and/or wherein step (b) expanding further comprises contacting the NK lineage cells with an expansion composition comprising nicotinamide.

53. The method of claim 45, wherein step (b) expanding further comprises contacting the NK lineage cells with a small molecule AhR inhibitor, thereby modulating the NK lineage cell activation.

54. The method of claim 53, wherein the small molecule AhR inhibitor comprises CHIR223191, UM729, UM171, or SR1.
